SEQUENCE LISTING (6 sequences)

SEQ ID NO: 1 - DNA, 22 bp, synthetic construct
  source 1..22: mol_type = other DNA
cagagagggagaaagaaggtag

SEQ ID NO: 2 - DNA, 25 bp, synthetic construct
  source 1..25: mol_type = other DNA
cataagttgttgaagtgaggtagag

SEQ ID NO: 3 - DNA, 22 bp, synthetic construct
  source 1..22: mol_type = other DNA
catcagcaggacacgtcatcat

SEQ ID NO: 4 - DNA, 20 bp, synthetic construct
  source 1..20: mol_type = other DNA
tagatggccacaggactaga

SEQ ID NO: 5 - DNA, 20 bp, synthetic construct
  source 1..20: mol_type = other DNA
tgagcacttcagggatttga

SEQ ID NO: 6 - DNA, 23 bp, synthetic construct
  source 1..23: mol_type = other DNA
cgtctgctagttctttactggcc

(19) United States
(12) Patent Application Publication
(10) Pub. No.: US 2025/0295638 A1
(43) Pub. Date: Sep. 25, 2025

(54) METHODS OF TREATING ALLOGRAFT REJECTION

(71) Applicant: Bargent Therapeutics Pty Ltd, Adamstown, NSW (AU)

(72) Inventor: Adrian Hibberd, Newcastle, New South Wales (AU)

(21) Appl. No.: 18/863,534

(22) PCT Filed: Apr. 22, 2023
(86) PCT No.: PCT/AU2023/050332

§ 371 Date: Nov. 6, 2024

(30) Foreign Application Priority Data
May 6, 2022 (AU) 2022901211
Sep. 15, 2022 (AU) 2022902674

(51) Int. Cl. A61K 31/423; A61K 31/436; A61K 38/13; A61K 45/06; A61P 37/06

(52) U.S. Cl. CPC A61K 31/423; A61K 31/436; A61K 38/13; A61K 45/06; A61P 37/06

(57) ABSTRACT

The present disclosure generally relates to methods of inhibiting an immune response and an immune response involved in transplant rejection, such as an allograft transplant rejection. In particular, the invention relates to the use of specific enzyme inhibitors that can be used to treat transplant rejection and/or prolong the survival of transplanted tissue or organs, in particular allotransplanted tissue or organs.

### TECHNICAL FIELD

The present disclosure generally relates to methods of inhibiting an immune response and an immune response involved in transplant rejection, such as an allotransplant rejection (or allograft rejection). In particular, the invention relates to the use of specific enzyme inhibitors for treating allotransplant rejection and/or prolonging the survival of allotransplanted tissue or organs.

### BACKGROUND

Any references to methods, apparatus or documents of the prior art are not to be taken as constituting any evidence or admission that they formed, or form part of the common general knowledge.

The preferred treatment for end-stage organ failure is organ transplantation. Although transplantation of various organs such as kidney, liver, heart, lung, heart-lung offer potential curative treatments for subjects presenting with organ failure, the suitability of recipients, a shortage of donors and failure of transplanted organ function, for example via immune rejection, all represent serious limitations on long-term success.

Despite significant advances in the understanding of transplant immunology, organ rejection remains the most substantial obstacle to both successful short-term and long-term organ transplantation. The following are key facts about the immune response of allotransplant rejection: initially recipient macrophages recognize foreign antigens on the allotransplant; they release cytokines and chemokines that activate the endothelial cells lining blood vessels and T cells thus facilitating alloreactive cell adhesion and entry through the vascular basement membrane of the allotransplant; T cells multiply and activate B cells governing alloantibody production; both T cells and alloantibodies damage the allotransplant because they recognize its foreignness which is conferred by antigens derived from the recipient's major histocompatibility complex (MHC). There are additional immune cell types (e.g., eosinophils, macrophages, mast cells) that can damage the allotransplant if the T/B cell lines are controlled by immunosuppressive agents. Hence, primary immune rejection response is adaptive and specific, recruiting T and B cells but the secondary responses are innate using macrophages and other like cell types. This damage is allotransplant rejection which can be acute or chronic, or both.

In general, a transplant immunosuppressive agent (or immunosuppressant) is a drug that reduces the ability of the recipient to reject the transplant defined by its ability to prolong organ allotransplant survival (compared with non-treated controls) and an immunological mechanism that facilitates reduction in allotransplant rejection.

The immune response involved with allotransplant rejection, and in particular, organ allotransplant rejection is known to be a very specific immune response that differs from other immune responses, for example an autoimmune response (which includes the immune response associated with insulin dependent diabetes mellitus) and an immune response initiated by an ischaemia reperfusion injury. Autoimmune rejection differs from allotransplant rejection in the following ways: autoimmune rejection is specific for autoantigens (recognized as self); not alloantigens recognized as non-self; it may cease naturally (for example polymyalgia rheumatica, rheumatoid arthritis) compared with allotransplant rejection which does not cease naturally; autoimmune rejection tends to be weaker than allotransplant rejection which can recruit other cell types if T cell mediated rejection is controlled by immunosuppressive agents. Further, autoimmune and allotransplant rejection have different mechanisms: allotransplant rejection has a rejecting arm in response to foreign (non-self) antigens introduced on the allotransplant, whereas autoimmune rejection results from breakdown in self-immunoregulatory mechanisms designed to accept self-antigens. Hence, any assumption that autoimmune rejection is equivalent to allotransplant rejection is flawed (1).

Reperfusion injury, also known as ischaemia-reperfusion injury (IRI) or reoxygenation injury, is tissue damage caused when blood supply returns to tissue after a period of ischaemia or lack of oxygen (anoxia or hypoxia). In the context of transplantation, ischaemia reperfusion injury (IRI) differs from allotransplant rejection in the following ways: IRI is an injury to an organ (or tissue) due to oxygen deficiency, lack of blood supply, reperfusion and resultant toxic products (radical oxygen species) when an organ (or tissue) is removed from a donor and implanted into a recipient and reperfused. By contrast, allotransplant rejection is a host response to foreign antigen on an allotransplant. Further, the recipient response to IRI mediated by innate immune cells (neutrophils, eosinophils, macrophages) is reversible without immunosuppression, whereas allotransplant rejection is usually mediated by T cells and antibodies and is never reversible without immunosuppression. Hence, the assumption that IRI is equivalent to allotransplant rejection is also flawed.

Roneparstat, a heparanase inhibitor, has been shown to curtail ischaemia reperfusion injury (2). However, reduction in IRI by heparanase inhibitors (2, 3, 4) should not be confused with treatment for allotransplant rejection.

In WO 2011/109877 (The Australian National University 15 Sep. 2011) (5) the authors claim that the survivals of pancreatic islet allografts in mice are prolonged by a heparan sulphate mimetic PI 88 (Example 7 and FIG. 15). It is stated that the mimetic acts by restoring the heparan sulphate content of islets with destructive insulitis, compared to saline treated control mice which exhibit substantial loss of islet heparan sulphate in the presence of destructive insulitis. However, there is no control arm for IRI which would consist of a group of mice with isograft islets treated with the same heparanase inhibitor (PI 88). Without this control the improvement in islet allograft survival is more consistent with control of IRI not control of islet allograft rejection. To this point, the disclosure of WO 2011/109877 postulates that heparan sulphate (or a heparan sulphate mimetic) inhibits oxidative damage which is a hallmark of IRI.

In WO 2008/046162 (The Australian National University 24 Apr. 2008) (6), the authors claim that heparan sulphate mimetic PI 88 curtails rejection of islet allografts in mice (example 8 and FIG. 10) by combatting the breakdown of the islet BM/ECM (basement membrane/extracellular matrix) heparan sulphate by heparanase. As such, this study is about preservation of engrafted allogeneic islets cells and not prolonged survival of allotransplant islets. Again, similarly to the disclosure of WO 2011/109877, there is no control in WO 2008/046162 for IRI. As such, the disclosure of less destruction of islets allografts in WO 2008/046162 could be due to the effect of the heparan sulphate mimetic on IRI alone (as subsequently alluded to in WO 2011/109877). Further, although WO 2008/046162 discloses a long list of heparanase inhibitors, there is nothing in WO 2008/046162 to support the use of those structurally distinct inhibitors in treating allotransplant rejection. Importantly, islet allotransplants are secondarily vascularized transplants not primarily vascularized transplants. Secondarily vascularized transplants are cellular transplants that do not have vascular anastomoses but are usually lodged into another organ or under its capsule using catheters lodged into the recipient's veins. In contrast, primarily vascularized transplants are organs that are connected to the recipient's blood supply by surgical arterial and venous anastomoses (linkages) and gain an immediate blood supply. Accordingly, there is no justification that a factor that works for secondarily vascularized transplants will necessarily work for primarily vascularized transplants given the higher risk of immediate antibody-mediated rejection in primarily vascularized transplants.

Heparanase is a complex multi-functional enzyme. It mediates, for example, proliferative diseases, autoimmune disease, psoriasis, macular degeneration and diabetes. Heparanase has been predominantly viewed as a promising target for cancer treatment for almost two decades as it is highly expressed in various cancers and its increased expression is associated with metastasis and increased tumour size (7).

More recently studies have identified heparanase as being involved in a range of pathologies in addition to cancer, including diabetes, bone necrosis, liver fibrosis, amyloidosis and Alzheimer's disease, and in the infection and spread of numerous viruses (7).

Although heparanase has been shown to stimulate the release of pro-inflammatory cytokines [interleukin IL-1B, IL-6, IL-8, IL-10, and tumour necrosis factor (TNF)-α] from peripheral blood mononuclear cells, its role, if any, in transplant rejection is currently unknown.

Previous studies of secondarily vascularized transplants using heparin derivatives have not established a role for heparanase inhibitors (8, 9) because none of the agents was a specific inhibitor of heparanase, controls for IRI were not included and other mechanisms could explain prolongation. For example, heparin could have worked by inhibition of tumour necrosis factor α (10) or as an anti-coagulant. In Lider et al (8) and Cohen et al (9) the authors have no control for IRI in models of skin allotransplants in mice: the prolongation of skin transplant survival could have influenced by the inhibition of IRI. Furthermore, skin allografts being secondarily vascularized behave differently from primarily vascularized transplants (the distinction between these noted above). These data do not establish that heparanase inhibitors control rejection of skin allografts nor by inference primarily vascularized transplants.

Despite the availability of clinical immunosuppressive treatments for transplant rejection, there remains a need for more efficacious treatments for transplant rejection. For example, there remains a need for treatments that exhibit low toxicity and are capable of long-term transplant immunosuppression.

It is an object of the present invention to overcome or ameliorate at least one of the disadvantages of the prior art, or to provide a useful alternative.

### SUMMARY

It has been surprisingly found that specific inhibition of heparanase activity prolongs allograft survival in transplant subjects. It has also unexpectedly been found that inhibition of heparanase activity is useful as a means of preventing and treating transplant rejection and/or maintaining allograft integrity.

It has also been unexpectedly found that systemic administration of heparanase inhibitors reduced the expression levels of heparanase in lymphocytes isolated from a transplant organ. Surprisingly, this reduction in heparanase expression levels was not observed in lymphocytes isolated from the peripheral blood of heparanase inhibitor treated transplant animals. Heparanase is known to be a complex multi-functional enzyme involved in many systems and processes in the body. Therefore, the results presented herein suggest that the treatment of transplant rejection by heparanase inhibitors is unlikely to significantly affect the wide spectrum of biological systems and processes in which heparanase may play a role, other than an immune allograft rejection response at the site of a transplant. It follows that the treatment of transplant rejection by administration of heparanase inhibitors is, for example, unlikely to have significant side effects or toxicity.

Thus, in one aspect, the present invention relates to a method of preventing or treating transplant rejection said method comprising the step of administering to a subject in need thereof a heparanase inhibitor, wherein said heparanase inhibitor inhibits heparanase activity and thereby prevents or treats transplant rejection.

In certain embodiments, the method of the invention includes treatment of transplant rejection where the rejection is of a primarily vascularized organ transplant. Preferably, the method of the invention treats or prevents transplant rejection of, for example but not limited to, a heart, heart-lung, kidney, liver, pancreas, stomach or intestine transplant. As such, the method of the invention excludes the treatment of secondarily vascularized transplants (such as islet beta cell transplants).

In one embodiment, the method of the invention, relates to the prevention or treatment of an allograft transplant rejection.

In a particular embodiment, the heparanase inhibitor used in the method of the invention is a benzoxazole, benzothiazole or benzimidazole acid derivative, as described in, for example, WO2004/046122, the contents of which are incorporated by reference. In a further embodiment the heparanase inhibitor is a benzoxazol-5-yl acetic acid derivative. In yet a further embodiment, the heparanase inhibitor is OGT 2115 having the formula,

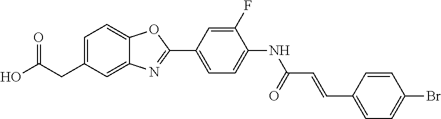 [SMILES: O=C(O)Cc1ccc2oc(-c3ccc(NC(=O)/C=C/c4ccc(Br)cc4)c(F)c3)nc2c1]

or a functional derivative or functional analogue thereof.

OGT 2115 is a benzoxazol-5-yl acetic acid derivative also known as 2-[4-[3-(4-Bromophenyl)-1-oxo-2-propenyl] amino]-3-fluorophenyl]-5-benzoxazoleacetic acid and is a cell-permeable heparanase inhibitor.

The person of skill in the art would understand that OGT 2115 is commercially available, for example from MCE Med Chem Express and R&D Systems. Further methods of preparing benzoxazole, benzothiazole and benzimidazole acid derivative heparanase inhibitors, including OGT 2115, are described in, for example, WO2004046122, the contents of which are incorporated by reference.

A range of heparanase inhibitors would be known to the skilled person as being suitable for use in the method of the invention. For example, quinazoline compounds such as the ones described in WO2018107200 and WO2018107201, the contents of which are incorporated by reference. Such heparanase inhibitors include a compound of general Formula A

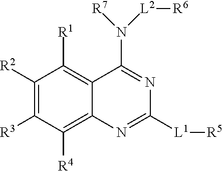

or a salt, hydrate, solvate, tautomer or stereoisomer thereof,
wherein:
R¹ is selected from H, hydroxyl, halo, C₁₋₆ alkyl, C₁₋₄ alkoxy, O-CH₂phenyl, O-phenyl;
R² is selected from H, hydroxyl, halo, C₁₋₆ alkyl, C₁₋₄ alkoxy, O-CH₂phenyl, O-phenyl;
R³ is selected from H, hydroxyl, halo, C₁₋₆ alkyl, C₁₋₄ alkoxy, O-CH₂phenyl, O-phenyl;
R⁴ is selected from H, hydroxyl, halo, C₁₋₆ alkyl, C₁₋₄ alkoxy, O-CH₂phenyl, O-phenyl;
or R¹ and R², or R² and R³, or R³ and R⁴ together form C₁₋₃alkylenedioxy;
wherein at least one of R¹, R², R³ and R⁴ is not H,
L¹ is selected from NHC₁₋₄alkyl-NHC(O)—, NHC₁₋₄alkyl-NHSO₂—, azetidinyl-NHC(O)—, and azetidinyl-NHSO₂—;
R⁵ is selected from C₃₋₉ cycloalkyl, C₆₋₁₀ aryl optionally substituted with 1 or 2 RX groups, C₂₋₉heteroaryl optionally substituted with 1 or 2 RX groups, C₂₋₅ heterocycloalkyl optionally substituted with 1 or 2 RX groups, C₁₋₄ alkylC₂₋₅ heterocycloalkyl optionally substituted with 1 or 2 RX groups;
L² is selected from C₁₋₄ alkyl, azetidinyl-C(O)—, C₁₋₄ alkyl-NHC(O)—, C₁₋₄ alkyl-NHSO₂—, —C(O)—, SO₂—; or L² is absent;
R⁶ is selected from H, C₂₋₆ alkyl, guanidinyl, NHC(NH)NH(C₁₋₃ alkyl), ureido, NHC(O)NH(C₁₋₃ alkyl), C₆₋₁₀ aryl optionally substituted with 1 or 2 RX groups, C₁₋₉heteroaryl optionally substituted with 1 or 2 RX groups, C₂₋₅ heterocycloalkyl optionally substituted with 1 or 2 RX groups, C₃₋₉ cycloalkyl optionally substituted with 1 or 2 RX groups;
R⁷ is H or C₁₋₆ alkyl;
and wherein when L¹ is NHC₁₋₄alkyl-NHSO₂—, R⁵ is not phenyl substituted with one methyl, tert-butyl or phenyl group;
each RX is independently selected from hydroxyl, halo, nitro, NR′R″ (wherein R′ and R″ are independently selected from H and C₁₋₃ alkyl), C₁₋₄ alkyl, C₃-9 cycloalkyl, haloC₁₋₄ alkyl, C₁₋₄alkoxy, C(O)C₁₋₃ alkyl, C(O)OC₁₋₄ alkyl, C(O)NHRY, C₆₋₁₀ aryl optionally substituted with 1 or 2 RY groups, C₂₋₉ heteroaryl optionally substituted with 1 or 2 RY groups, C₁₋₄ alkyl-(C₂₋₉heteroaryl), C₂₋₅ heterocycloalkyl optionally substituted with 1 or 2 C₁₋₄ alkyl groups, C₁₋₄ alkyl-(C₂₋₅ heterocycloalkyl) optionally substituted with 1 or 2 C₁₋₄ alkyl groups, C(O)—C₂₋₉ heteroaryl optionally substituted with 1 or 2 C₁₋₄ alkyl groups; SO2-C₂₋₉ heteroaryl optionally substituted with 1 or 2 C₁₋₄ alkyl groups, or haloC₁₋₄ alkyl groups; or two adjacent RX groups together form C₁₋₃ alkylenedioxy;
RY is selected from H, hydroxyl, halo, C₁₋₄ alkyl, haloC₁₋₄alkyl, C₁₋₄ alkoxy.

Heparanase inhibitors also include a compound of general Formula I

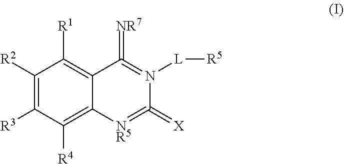

or a salt, hydrate, solvate, tautomer or stereoisomer thereof,
wherein:
X is S or O;
R¹ is selected from H, hydroxyl, halo, C₁₋₆alkyl, C₁₋₄alkoxy, O—CH₂phenyl, O-phenyl;
R² is selected from H, hydroxyl, halo, C₁₋₆alkyl, C₁₋₄alkoxy, O—CH₂phenyl, O-phenyl;
R³ is selected from H, hydroxyl, halo, C₁₋₆alkyl, C₁₋₄alkoxy, O—CH₂phenyl, O-phenyl;
R⁴ is selected from H, hydroxyl, halo, C₁₋₆alkyl, C₁₋₄alkoxy, O—CH₂phenyl, O-phenyl;
or R¹ and R², or R² and R³, or R³ and R⁴ together form C₁₋₃alkylenedioxy;
R⁵ is selected from H, C₁₋₆ alkyl, C₁₋₃alkylC(O)OC₁₋₄alkyl and C₁₋₃alkylC₆₋₁₀aryl optionally substituted with 1 or 2 groups independently selected from haloC₁₋₃alkyl and haloC₁₋₃alkoxy;
L is selected from C₁₋₆ alkyl, azetidinyl, C₁₋₆alkyl-indolyl, NH, C₁₋₆alkyl-NI—IC(O) 0, azetidinyl-C(O)—, C₁₋₆alkyl-NHC(O)-indolyl, C₁₋₆alkyl-NHSO₂—, or is absent;
R⁶ is selected from H, halo, hydroxyl, d.₆alkyl, C₁₋₆alkenyl, C₁₋₆alkynyl, C₆₋₁₀aryl optionally substituted with 1 or 2 R^{x} groups, C₁₋₉ heteroaryl optionally substituted with 1 or 2 R^{x} groups, C₂₋₅ heterocycloalkyl optionally substituted with 1 or 2 R^{x} groups, C(O)-(heterocycloalkyl) optionally substituted with 1 or 2 R^{x} groups, C(O)(C_{2.5}heterocycloalkyl) optionally substituted with 1 or 2 R^{x} groups, C(O)NHR^{Y}, or is absent;
each R^{x} is independently selected from hydroxyl, halo, nitro, NR′R″, C₁₋₄alkyl, C₃₋₆ cycloalkyl, haloC₁₋₄alkyl, C₁₋₄alkoxy, C₆₋₁₀aryl optionally substituted with 1 or 2 R^{Y} groups, C₁₋₉heteroaryl, C₁₋₄alkyl-(C₁₋₉heteroaryl), C(O)OC₁₋₄ alkyl, C(O)NHR^{Y}, C₂₋₅heterocycloalkyl optionally substituted with 1 or 2 C₁₋₄alkyl groups, C(O)-(heterocycloalkyl) optionally substituted with 1 or 2 C₁₋₄alkyl groups, or two adjacent R^{x} groups together form C₁₋₃alkylenedioxy;
R^{Y} is selected from H, hydroxyl, halo, C₁₋₄ alkyl, haloC₁₋₄ alkyl, C₁₋₄ alkoxy,
C₁₋₄ alkylheterocycloalkyl, C(O)—(C₁₋₄ alkylheterocycloalkyl), C₁₋₄alkyINR′R″;
R′ and R″ are independently selected from H, C₁₋₄alkyl, C₁₋₄ alkylheterocycloalkyl;
R⁷ is selected from H, C₁₋₄ alkyl and C₁₋₆alkylC₁₋₉ heteroaryl.

Heparanase inhibitors also include a compound of general Formula II

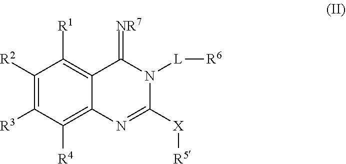

or a salt, hydrate, solvate, tautomer or stereoisomer thereof,
wherein:
X is S or O;
R¹ is selected from H, hydroxyl, halo, C₁₋₆alkyl, C₁₋₄alkoxy, O—CH₂phenyl, O-phenyl;
R² is selected from H, hydroxyl, halo, C₁₋₆alkyl, C₁₋₄alkoxy, O—CH₂phenyl, O-phenyl;
R³ is selected from H, hydroxyl, halo, C₁₋₆alkyl, C₁₋₄alkoxy, O—CH₂phenyl, O-phenyl;
R⁴ is selected from H, hydroxyl, halo, C₁₋₆alkyl, C₁₋₄alkoxy, O—CH₂phenyl, O-phenyl;
or R¹ and R², or R² and R³, or R³ and R⁴ together form C₁₋₃alkylenedioxy;
R⁵′ is selected from H, C₁₋₆ alkyl, C₁₋₃alkylC\hat{ }OC₁₋₄alkyl and C₁₋₃alkylC₆₋₁₀aryl optionally substituted with 1 or 2 groups independently selected from haloC₁₋₃-alkyl and haloC₁₋₃alkoxy;
L is selected from C₁₋₆ alkyl, azetidinyl, C₁₋₆alkyl-indolyl, NH, C₁₋₆alkyl-NI—IC(O) 0, azetidinyl-C(O)—, C₁₋₆alkyl-NHC(O)-indolyl, C₁₋₆alkyl-NHSO₂—, or is absent;
R⁶ is selected from H, halo, hydroxyl, C₁₋₆alkyl, C₁₋₆alkenyl, C₁₋₆alkynyl, C₆₋₁₀aryl optionally substituted with 1 or 2 R^{x} groups, 0₁₋₉ heteroaryl optionally substituted with 1 or 2 R^{x} groups, C₂₋₅ heterocycloalkyl optionally substituted with 1 or 2 R^{x} groups, C(O)-(heterocycloalkyl) optionally substituted with 1 or 2 R^{x} groups, C(O)(C_{2.5}heterocycloalkyl) optionally substituted with 1 or 2 R^{x} groups, C(O)NHR^{Y}, or is absent;
each R^{x} is independently selected from hydroxyl, halo, nitro, NR′R″, C₁₋₄alkyl, C₃₋₆cycloalkyl, haloC₁₋₄alkyl, C₁₋₄alkoxy, C₆₋₁₀aryl optionally substituted with 1 or 2 R^{Y} groups, C₁₋₉heteroaryl, C₁₋₄alkyl-id-gheteroaryl), O(O)OO_{!-4} alkyl, C(O)NHR^{Y}, C₂₋₅heterocycloalkyl optionally substituted with 1 or 2 C₁₋₄alkyl groups, C(O)-(heterocycloalkyl) optionally substituted with 1 or 2 C₁₋₄alkyl groups, or two adjacent R^{x} groups together form C₁₋₃alkylenedioxy;
R^{Y} is selected from H, hydroxyl, halo, C₁₋₄ alkyl, haloC₁₋₄ alkyl, C₁₋₄ alkoxy, C₁₋₄ alkylheterocycloalkyl, C(O)—(C₁₋₄ alkylheterocycloalkyl), C₁₋₄alkyINR′R″;
R′ and R″ are independently selected from H, C₁₋₄alkyl, C₁₋₄ alkylheterocycloalkyl;
R⁷ is selected from H, C₁₋₄ alkyl and C₁₋₆alkylC₁₋₉ heteroaryl.

Heparanase inhibitors also include a compound of general Formula III

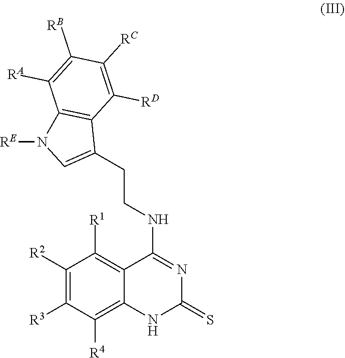

wherein groups R¹, R², R³, and R⁴ are as defined for formulae (I) and (II);
R^{A}, R^{B}, R^{c} and R^{D} are independently selected from H, OH, C₁₋₃ alkyl, 00₁₋₃ alkyl, C(O)—(N-heterocycloalkyl) (e.g., C(O)morpholinyl), C(O)piperazinyl) optionally substituted with 1 or 2 C₁₋₃ alkyl groups; N-heteroaryl (e.g., 3-pyridyl, 4-pyridyl, 2,1,3-benzoxadiazolyl, pyrazolyl) optionally substituted with 1 or 2 groups selected from OH, halo, C₁₋₃ alkyl, OC₁₋₃ alkyl; phenyl optionally substituted with 1 or 2 groups selected from OH, halo, C₁₋₃ alkyl, OC₁₋₃ alkyl,
CiOJNHC₁₋₃alkyKNiC₁₋₃alkyl\hat{ }], C(O) (heterocycloalkyl) (e.g., morpholinyl, piperazinyl, piperidinyl) optionally substituted with 1 or 2 C₁₋₃ alkyl groups;
R^{E} is H, C₁₋₃alkyl, or C(O)-heterocycloalkyl (e.g., C(O)—(N-morpholino)).

The skilled person would readily understand how to produce the heparanase inhibitors of Formula A, Formula I, Formula II and Formula III from the disclosures of WO2018107200 and WO2018107201, which are incorporated by reference.

In another embodiment the present invention related to the use of monoclonal or polyclonal antibodies that target heparanase in the allotransplant at induction of treatment or for rejection or for chronic allotransplant rejection.

In a further embodiment, the present invention relates to a method of preventing or treating allotransplant rejection said method comprising the step of administering to a subject in need thereof a heparanase inhibitor, wherein said heparanase inhibitor reduces the level of heparanase activity and thereby prevents or treats transplant rejection.

In a further embodiment, the present invention relates to a method of preventing or treating transplant rejection said method comprising the step of administering to a subject in need thereof a heparanase inhibitor, wherein said heparanase inhibitor reduces the expression level of heparanase in lymphocytes and thereby prevents or treats allotransplant rejection. Preferably, the reduction in expression levels of heparanase occurs in lymphocytes at the allotransplant site, for example at an organ allotransplant site.

In a particularly preferred embodiment, the present invention relates to a method of preventing or treating transplant rejection said method comprising the step of administering to a subject in need thereof a heparanase inhibitor, wherein said heparanase inhibitor reduces the expression level of heparanase in lymphocytes at a transplant site, for example a transplanted organ, and wherein the expression levels of heparanase are not reduced in lymphocytes in the peripheral blood of the subject. In one embodiment, the heparanase inhibitor administered to the subject to reduce heparanase expression levels in lymphocytes at a transplant site is selected from OGT 2115 or Castanospermine. Both OGT 2115 and Castanospermine have been shown by the present inventor to selectively reduce expression levels of heparanase in lymphocytes at a transplant site, such as the transplanted organ site.

The skilled person would understand from the present invention that any heparanase inhibitor capable of reducing heparanase expression levels would be suitable for the method of the present invention. The skilled person would also understand that it would be a matter of routine to identify heparanase inhibitors capable of reducing expression levels in lymphocytes.

In a particular embodiment, the method of the invention comprises administering the heparanase inhibitor in combination with one or more immunosuppressive drugs. Suitable immunosuppressive drugs would be well known to the person of skill in the relevant art. Preferably, the one or more immunosuppressive drugs may be selected from the group consisting of methotrexate, mizoribine, cyclosporin, aerosolized cyclosporin, tacrolimus, mycophenolate mofetil, azathioprine, sirolimus and other mTOR inhibitors, deoxyspergualin, leflunomide, malononitriloamide analogues of leflunomide; anti-CTLA4 antibodies, anti-CTLA4 Ig fusions, anti-B lymphocyte stimulator antibodies, anti-CD80 antibodies, etanercept, infliximab, anti-T cell antibodies, anti-CD3 antibodies, OKT3, anti-CD4 antibodies, anti il-2 receptor antibodies, prednisolone or its derivatives, anti CD52 monoclonal antibodies; anti-CD20 monoclonal antibodies; belatacept; eculizumab; and intravenous immunoglobulin.

In a particular embodiment, the immunosuppressive drug is cyclosporin A or tacrolimus that are in routine use for maintenance immunosuppression treatment for organ transplant recipients.

In a further embodiment, the method of the invention comprises administering the heparanase inhibitor in combination with one or more anti-inflammatory drugs. Suitable anti-inflammatory drugs would be well known to the person of skill in the relevant art. Preferably, the one or more immunosuppressive drugs may be selected from the group consisting of corticosteroids, clobetasol, halobetasol, hydrocortisone, triamcinolone, betamethasone, fluocinolone, fluocinonide, prednisone, prednisolone and methylprednisolone.

In a further embodiment, the present invention relates to a method of preventing or treating an allotransplant rejection said method comprising the step of administering to a subject in need thereof OGT 2115 of formula

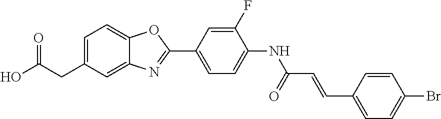 [SMILES: O=C(O)Cc1ccc2oc(-c3ccc(NC(=O)/C=C/c4ccc(Br)cc4)c(F)c3)nc2c1]

or a functional derivative or analogue thereof, wherein said OGT 2115 inhibits heparanase activity and thereby prevents or treats transplant rejection.

In another embodiment, the present invention relates to a method of preventing or treating an allograft transplant rejection said method comprising the step of administering to a subject in need thereof OGT 2115 of formula

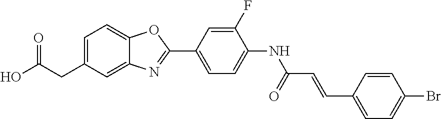 [SMILES: O=C(O)Cc1ccc2oc(-c3ccc(NC(=O)/C=C/c4ccc(Br)cc4)c(F)c3)nc2c1]

or a functional derivative or analogue thereof, in combination with cyclosporin A or tacrolimus wherein said OGT2115 inhibits heparanase and thereby prevents or treats transplant rejection.

In another embodiment, in the method of the invention, the heparanase inhibitor is administered to a subject in therapeutically effective dose. In another embodiment, in the method of the invention, the heparanase inhibitor is administered to a subject in a dose from 1 mg/kg to 50 mg/kg. Specifically, the heparanase inhibitor is administered at a dosage of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg. In a further embodiment the heparanase inhibitor is administered at a dosage of 2.0 mg/kg to 10 mg/kg. Specifically, this includes 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0, 6.5 mg/kg, 7.0 mg/kg, 7.5 mg/kg, 8.0 mg/kg, 8.5 mg/kg, 9.0 mg/kg, 9.5 mg/kg, or 10 mg/kg.

In the method of the invention, the heparanase inhibitor is administered to a subject at any one of the dosages described in paragraph above daily, for example once a day or twice a day. Alternatively, the heparanase inhibitor is administered weekly, for example once weekly.

When the heparanase inhibitor of the invention is administered in combination with a one or more immunosuppressive drugs, the immunosuppressive drugs, are administered in a therapeutically effective dose.

In a particular embodiment, OGT 2115 of formula

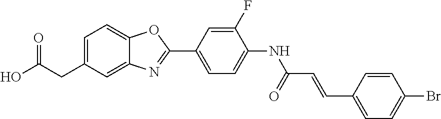 [SMILES: O=C(O)Cc1ccc2oc(-c3ccc(NC(=O)/C=C/c4ccc(Br)cc4)c(F)c3)nc2c1]

or a functional derivative or analog thereof is administered at a dosage of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg.

In a further embodiment the OGT 2115 of formula

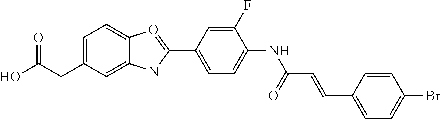

is administered at a dosage of 2.0 mg/kg to 10 mg/kg. Specifically, this includes 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0, 6.5 mg/kg, 7.0 mg/kg, 7.5 mg/kg, 8.0 mg/kg, 8.5 mg/kg, 9.0 mg/kg, 9.5 mg/kg, or 10 mg/kg.

In an embodiment of the method of the invention, OGT 2115 is administered to a subject at any one of the dosages described in paragraph above daily, for example once a day. Alternatively, the heparanase inhibitor is administered weekly, for example once weekly.

When OGT 2115 is administered in combination with a therapeutically effective dose of cyclosporin A or tacrolimus.

In a further embodiment, the present invention relates to a method of preventing or treating an allograft transplant rejection said method comprising the step of administering to a subject in need thereof a quinazoline compound.

In an alternative embodiment, the present invention relates to a method of preventing or treating an allograft transplant rejection said method comprising the step of administering to a subject in need thereof a compound of general Formula A

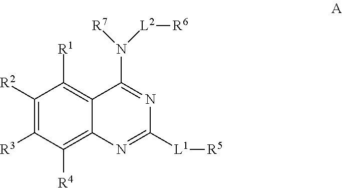

or a salt, hydrate, solvate, tautomer or stereoisomer thereof,
wherein:
R¹ is selected from H, hydroxyl, halo, C₁₋₆ alkyl, C₁₋₄ alkoxy, O-CH₂phenyl, O-phenyl;
R² is selected from H, hydroxyl, halo, C₁₋₆ alkyl, C₁₋₄ alkoxy, O-CH₂phenyl, O-phenyl;
R³ is selected from H, hydroxyl, halo, C₁₋₆ alkyl, C₁₋₄ alkoxy, O-CH₂phenyl, O-phenyl;
R⁴ is selected from H, hydroxyl, halo, C₁₋₆ alkyl, C₁₋₄ alkoxy, O-CH₂phenyl, O-phenyl;
or R¹ and R², or R² and R³, or R³ and R⁴ together form C₁₋₃alkylenedioxy;
wherein at least one of R¹, R², R³ and R⁴ is not H,
L¹ is selected from NHC₁₋₄alkyl-NHC(O)—, NHC₁₋₄alkyl-NHSO₂—, azetidinyl-NHC(O)—, and azetidinyl-NHSO₂—;
R⁵ is selected from C₃₋₉ cycloalkyl, C₆₋₁₀ aryl optionally substituted with 1 or 2 RX groups, C₂₋₉heteroaryl optionally substituted with 1 or 2 RX groups, C₂₋₅ heterocycloalkyl optionally substituted with 1 or 2 RX groups, C₁₋₄ alkylC₂₋₅ heterocycloalkyl optionally substituted with 1 or 2 RX groups;
L² is selected from C₁₋₄ alkyl, azetidinyl-C(O)—, C₁₋₄ alkyl-NHC(O)—, C₁₋₄ alkyl-NHSO₂—, —C(O)—, SO₂—; or L² is absent;
R⁶ is selected from H, C₂₋₆ alkyl, guanidinyl, NHC(NH)NH(C₁₋₃ alkyl), ureido, NHC(O)NH(C₁₋₃ alkyl), C₆₋₁₀ aryl optionally substituted with 1 or 2 RX groups, C₁₋₉ heteroaryl optionally substituted with 1 or 2 RX groups, C₂₋₅ heterocycloalkyl optionally substituted with 1 or 2 RX groups, C₃₋₉ cycloalkyl optionally substituted with 1 or 2 RX groups;
R⁷ is H or C₁₋₆ alkyl;
and wherein when L¹ is NHC₁₋₄alkyl-NHSO₂—, R⁵ is not phenyl substituted with one methyl, tert-butyl or phenyl group;
each RX is independently selected from hydroxyl, halo, nitro, NR′R″ (wherein R′ and R″ are independently selected from H and C₁₋₃ alkyl), C₁₋₄ alkyl, C₃-9 cycloalkyl, haloC₁₋₄ alkyl, C₁₋₄alkoxy, C(O)C₁₋₃ alkyl, C(O)OC₁₋₄ alkyl, C(O)NHRY, C₆₋₁₀ aryl optionally substituted with 1 or 2 RY groups, C₂₋₉ heteroaryl optionally substituted with 1 or 2 RY groups, C₁₋₄ alkyl-(C₂₋₉heteroaryl), C₂₋₅ heterocycloalkyl optionally substituted with 1 or 2 C₁₋₄ alkyl groups, C₁₋₄ alkyl-(C₂₋₅ heterocycloalkyl) optionally substituted with 1 or 2 C₁₋₄ alkyl groups, C(O)—C₂₋₉ heteroaryl optionally substituted with 1 or 2 C₁₋₄ alkyl groups; SO₂-C₂₋₉ heteroaryl optionally substituted with 1 or 2 C₁₋₄ alkyl groups, or haloC₁₋₄ alkyl groups; or two adjacent RX groups together form C₁₋₃ alkylenedioxy;
RY is selected from H, hydroxyl, halo, C₁₋₄ alkyl, haloC₁₋₄alkyl, C₁₋₄ alkoxy wherein said compound of Formula A or a salt, hydrate, solvate, tautomer or stereoisomer thereof inhibits heparanase and thereby prevents or treats transplant rejection.

In an alternative embodiment, the present invention relates to a method of preventing or treating an allograft transplant rejection said method comprising the step of administering to a subject in need thereof a compound of general Formula I

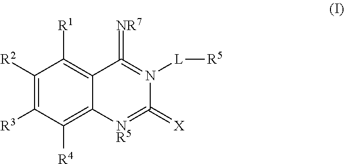

or a salt, hydrate, solvate, tautomer or stereoisomer thereof,
wherein:
X is S or O;
R¹ is selected from H, hydroxyl, halo, C₁₋₆alkyl, C₁₋₄alkoxy, O—CH₂phenyl, O-phenyl;
R² is selected from H, hydroxyl, halo, C₁₋₆alkyl, C₁₋₄alkoxy, O—CH₂phenyl, O-phenyl;
R³ is selected from H, hydroxyl, halo, C₁₋₆alkyl, C₁₋₄alkoxy, O—CH₂phenyl, O-phenyl;
R⁴ is selected from H, hydroxyl, halo, C₁₋₆alkyl, C₁₋₄alkoxy, O—CH₂phenyl, O-phenyl;
or R¹ and R², or R² and R³, or R³ and R⁴ together form C₁₋₃alkylenedioxy;
R⁵ is selected from H, C₁₋₆ alkyl, C₁₋₃alkylC(O)OC₁₋₄alkyl and C₁₋₃alkylC₆₋₁₀aryl optionally substituted with 1 or 2 groups independently selected from haloC₁₋₃alkyl and haloC₁₋₃alkoxy;
L is selected from C₁₋₆ alkyl, azetidinyl, C₁₋₆alkyl-indolyl, NH, C₁₋₆alkyl-NI—IC(O) 0, azetidinyl-C(O)—, C₁₋₆alkyl-NHC(O)-indolyl, C₁₋₆alkyl-NHSO₂—, or is absent;
R⁶ is selected from H, halo, hydroxyl, d.₆alkyl, C₁₋₆alkenyl, C₁₋₆alkynyl, C₆₋₁₀aryl optionally substituted with 1 or 2 R^{x} groups, C₁₋₉ heteroaryl optionally substituted with 1 or 2 R^{x} groups, C₂₋₅ heterocycloalkyl optionally substituted with 1 or 2 R^{x} groups, C(O)-(heterocycloalkyl) optionally substituted with 1 or 2 R^{x} groups, C(O)(C_{2.5}heterocycloalkyl) optionally substituted with 1 or 2 R^{x} groups, C(O)NHR^{Y}, or is absent;
each R^{x} is independently selected from hydroxyl, halo, nitro, NR′R″, C₁₋₄alkyl, C₃₋₆cycloalkyl, haloC₁₋₄alkyl, C₁₋₄alkoxy, C₆₋₁₀aryl optionally substituted with 1 or 2 R^{Y} groups, C₁₋₉heteroaryl, C₁₋₄alkyl-(C₁₋₉heteroaryl), C(O)OC₁₋₄ alkyl, C(O)NHR^{Y}, C₂₋₅heterocycloalkyl optionally substituted with 1 or 2 C₁₋₄alkyl groups, C(O)-(heterocycloalkyl) optionally substituted with 1 or 2 C₁₋₄alkyl groups, or two adjacent R^{x} groups together form C₁₋₃alkylenedioxy;
R^{Y} is selected from H, hydroxyl, halo, C₁₋₄ alkyl, haloC₁₋₄ alkyl, C₁₋₄ alkoxy,
C₁₋₄ alkylheterocycloalkyl, C(O)—(C₁₋₄ alkylheterocycloalkyl), C₁₋₄alkylNR′R″;
R′ and R″ are independently selected from H, C₁₋₄alkyl, C₁₋₄ alkylheterocycloalkyl;
R⁷ is selected from H, C₁₋₄ alkyl and C₁₋₆alkylC₁₋₉ heteroaryl.

In an alternative embodiment, the present invention relates to a method of preventing or treating an allograft transplant rejection said method comprising the step of administering to a subject in need thereof a compound of general Formula II

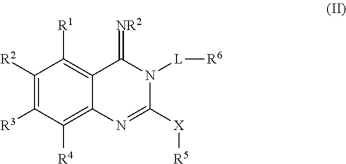

or a salt, hydrate, solvate, tautomer or stereoisomer thereof,
wherein:
X is S or O;
R¹ is selected from H, hydroxyl, halo, C₁₋₆alkyl, C₁₋₄alkoxy, O—CH₂phenyl, O-phenyl;
R² is selected from H, hydroxyl, halo, C₁₋₆alkyl, C₁₋₄alkoxy, O—CH₂phenyl, O-phenyl;
R³ is selected from H, hydroxyl, halo, C₁₋₆alkyl, C₁₋₄alkoxy, O—CH₂phenyl, O-phenyl;
R⁴ is selected from H, hydroxyl, halo, C₁₋₆alkyl, C₁₋₄alkoxy, O—CH₂phenyl, O-phenyl;
or R¹ and R², or R² and R³, or R³ and R⁴ together form C₁₋₃alkylenedioxy;
R⁵′ is selected from H, C₁₋₆ alkyl, C₁₋₃alkylC\hat{ }OC₁₋₄alkyl and C₁₋₃alkylC₆₋₁₀aryl optionally substituted with 1 or 2 groups independently selected from haloC₁₋₃alkyl and haloC₁₋₃alkoxy;
L is selected from C₁₋₆ alkyl, azetidinyl, C₁₋₆alkyl-indolyl, NH, C₁₋₆alkyl-NI—IC(O) 0, azetidinyl-C(O)—, C₁₋₆alkyl-NHC(O)-indolyl, C₁₋₆alkyl-NHSO₂—, or is absent;
R⁶ is selected from H, halo, hydroxyl, C₁₋₆alkyl, C₁₋₆alkenyl, C₁₋₆alkynyl, C₆₋₁₀aryl optionally substituted with 1 or 2 R^{x} groups, 0₁₋₉ heteroaryl optionally substituted with 1 or 2 R^{x} groups, C₂₋₅ heterocycloalkyl optionally substituted with 1 or 2 R^{x} groups, C(O)-(heterocycloalkyl) optionally substituted with 1 or 2 R^{x} groups, C(O)(C_{2.5}heterocycloalkyl) optionally substituted with 1 or 2 R^{x} groups, C(O)NHR^{Y}, or is absent;
each R^{x} is independently selected from hydroxyl, halo, nitro, NR′R″, C₁₋₄alkyl, C₃₋₆cycloalkyl, haloC₁₋₄alkyl, C₁₋₄alkoxy, C₆₋₁₀aryl optionally substituted with 1 or 2 R^{Y} groups, C₁₋₉heteroaryl, C₁₋₄alkyl-id-gheteroaryl), O(O)OO_{!-4} alkyl, C(O)NHR^{Y}, C₂₋₅heterocycloalkyl optionally substituted with 1 or 2 C₁₋₄alkyl groups, C(O)-(heterocycloalkyl) optionally substituted with 1 or 2 C₁₋₄alkyl groups, or two adjacent R^{x} groups together form C₁₋₃alkylenedioxy;
R^{Y} is selected from H, hydroxyl, halo, C₁₋₄ alkyl, haloC₁₋₄ alkyl, C₁₋₄ alkoxy, C₁₋₄ alkylheterocycloalkyl, C(O)—(C₁₋₄ alkylheterocycloalkyl), C₁₋₄alkyINR′R″;
R′ and R″ are independently selected from H, C₁₋₄alkyl, C₁₋₄ alkylheterocycloalkyl;
R⁷ is selected from H, C₁₋₄ alkyl and C₁₋₆alkylC₁₋₉ heteroaryl.

In a further embodiment, the present invention relates to a method of preventing or treating an allograft transplant rejection said method comprising the step of administering to a subject in need thereof a compound of general Formula III

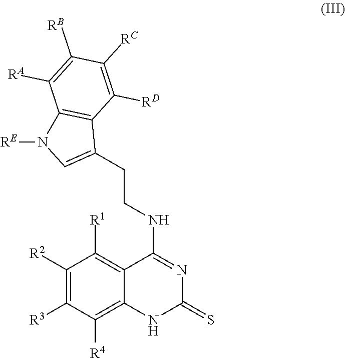

wherein groups R¹, R², R³, and R⁴ are as defined for formulae (I) and (II);
R^{A}, R^{B}, R^{c} and R^{D} are independently selected from H, OH, C₁₋₃ alkyl, 00₁₋₃ alkyl, C(O)—(N-heterocycloalkyl) (e.g., C(O)morpholinyl), C(O)piperazinyl) optionally substituted with 1 or 2 C₁₋₃ alkyl groups; N-heteroaryl (e.g., 3-pyridyl, 4-pyridyl, 2,1,3-benzoxadiazolyl, pyrazolyl) optionally substituted with 1 or 2 groups selected from OH, halo, C₁₋₃ alkyl, OC₁₋₃ alkyl; phenyl optionally substituted with 1 or 2 groups selected from OH, halo, C₁₋₃ alkyl, OC₁₋₃ alkyl,
CiOJNHC₁₋₃alkyKNiC₁₋₃alkyl\hat{ }], C(O) (heterocycloalkyl) (e.g., morpholinyl, piperazinyl, piperidinyl) optionally substituted with 1 or 2 C₁₋₃ alkyl groups;
R^{E} is H, C₁₋₃alkyl, or C(O)-heterocycloalkyl (e.g., C(O)—(N-morpholino)).

Preferably, for the embodiments directed to the method of the invention, prevention or treatment of transplant rejection comprises prolonging the survival time of the transplant.

In a further aspect, the present invention relates to the use of a heparanase inhibitor in the preparation of a medicament for the prevention or treatment of transplant rejection wherein said heparanase inhibitor inhibits heparanase activity and thereby prevents or treats transplant rejection.

In yet a further aspect, the present invention relates to the use of a heparanase inhibitor in the preparation of a medicament for the prevention or treatment of allotransplant rejection wherein said heparanase inhibitor reduces the expression level of heparanase and thereby prevents or treats transplant rejection.

In one embodiment, the invention relates to the use of a heparanase inhibitor in the preparation of a medicament for the prevention or treatment of allotransplant rejection wherein said heparanase inhibitor reduces the expression level of heparanase in lymphocytes at a transplant site, for example, a transplanted organ or tissue, and wherein expression levels of heparanase are not reduced in lymphocytes in the peripheral blood of the subject.

In a preferred embodiment, the present invention relates to the use of a heparanase inhibitor in the preparation of a medicament for the prevention or treatment of transplant rejection wherein said heparanase inhibitor reduces the expression level of heparanase in lymphocytes at a transplant site, for example, a transplanted organ or tissue, and wherein expression levels of heparanase are not reduced in lymphocytes in the peripheral blood of the subject.

In one embodiment of the invention, the heparanase inhibitor is selected from OGT 2115 or Castanospermine. The person skilled in the art would understand that both OGT 2115 and Castanospermine are compounds that are commercially available.

In one embodiment, the use of the present invention prevents or treats rejection of a primarily vascularized organ transplant (i.e. excluding secondarily vascularised transplants). Preferably, the use of the invention treats or prevents transplant rejection of, for example but not limited to, a heart, heat-lung, kidney, lung, liver, pancreas, stomach or intestine transplant.

In one embodiment, the use of the invention, relates to the prevention or treatment of an allograft transplant rejection.

In a certain embodiment, the present invention relates to use of a benzoxazol-5-yl acetic acid derivative in the preparation of a medicament for the prevention or treatment of transplant rejection wherein said benzoxazol-5-yl acetic acid derivative inhibits heparanase activity and thereby prevents or treats transplant rejection.

In a particular embodiment, the present invention relates to use of OGT 2115 having the formula,

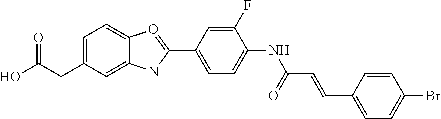

or a functional derivative or functional analog thereof in the preparation of a medicament for the prevention or treatment of transplant rejection wherein said OGT 2115 inhibits heparanase activity and thereby prevents or treats transplant rejection.

In an alternative embodiment the present invention relates to use of a quinazoline compound in the preparation of a medicament for the prevention or treatment of transplant rejection wherein said quinazoline compound inhibits heparanase activity and thereby prevents or treats transplant rejection.

In a specific embodiment, the present invention relates to use of a heparanase inhibitor in the preparation of a medicament for the prevention or treatment of transplant rejection wherein said heparanase inhibitor inhibits heparanase activity and thereby prevents or treats transplant rejection and wherein said heparanase inhibitor is a compound of general Formula A

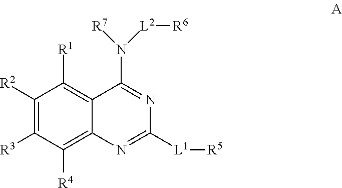

or a salt, hydrate, solvate, tautomer or stereoisomer thereof,
wherein:
R¹ is selected from H, hydroxyl, halo, C₁₋₆ alkyl, C₁₋₄ alkoxy, O-CH₂phenyl, O-phenyl;
R² is selected from H, hydroxyl, halo, C₁₋₆ alkyl, C₁₋₄ alkoxy, O-CH₂phenyl, O-phenyl;
R³ is selected from H, hydroxyl, halo, C₁₋₆ alkyl, C₁₋₄ alkoxy, O-CH₂phenyl, O-phenyl;
R⁴ is selected from H, hydroxyl, halo, C₁₋₆ alkyl, C₁₋₄ alkoxy, O-CH₂phenyl, O-phenyl;
or R¹ and R², or R² and R³, or R³ and R⁴ together form C₁₋₃alkylenedioxy;
wherein at least one of R¹, R², R³ and R⁴ is not H,
L¹ is selected from NHC₁₋₄alkyl-NHC(O)—, NHC₁₋₄alkyl-NHSO₂—, azetidinyl-NHC(O)—, and azetidinyl-NHSO₂—;
R⁵ is selected from C₃₋₉ cycloalkyl, C₆₋₁₀ aryl optionally substituted with 1 or 2 RX groups, C₂₋₉heteroaryl optionally substituted with 1 or 2 RX groups, C₂₋₅ heterocycloalkyl optionally substituted with 1 or 2 RX groups, C₁₋₄ alkylC₂₋₅ heterocycloalkyl optionally substituted with 1 or 2 RX groups;
L² is selected from C₁₋₄ alkyl, azetidinyl-C(O)—, C₁₋₄ alkyl-NHC(O)—, C₁₋₄ alkyl-NHSO₂—, —C(O)—, SO₂—; or L² is absent;
R⁶ is selected from H, C₂₋₆ alkyl, guanidinyl, NHC(NH)NH(C₁₋₃ alkyl), ureido, NHC(O)NH(C₁₋₃ alkyl), C₆₋₁₀ aryl optionally substituted with 1 or 2 RX groups, C₁₋₉heteroaryl optionally substituted with 1 or 2 RX groups, C₂₋₅ heterocycloalkyl optionally substituted with 1 or 2 RX groups, C₃₋₉ cycloalkyl optionally substituted with 1 or 2 RX groups;
R⁷ is H or C₁₋₆ alkyl;
and wherein when L¹ is NHC₁₋₄alkyl-NHSO₂—, R⁵ is not phenyl substituted with one methyl, tert-butyl or phenyl group;
each RX is independently selected from hydroxyl, halo, nitro, NR′R″ (wherein R′ and R″ are independently selected from H and C₁₋₃ alkyl), C₁₋₄ alkyl, C₃-9 cycloalkyl, haloC₁₋₄ alkyl, C₁₋₄alkoxy, C(O)C₁₋₃ alkyl, C(O)OC₁₋₄ alkyl, C(O)NHRY, C₆₋₁₀ aryl optionally substituted with 1 or 2 RY groups, C₂₋₉ heteroaryl optionally substituted with 1 or 2 RY groups, C₁₋₄ alkyl-(C₂₋₉heteroaryl), C₂₋₅ heterocycloalkyl optionally substituted with 1 or 2 C₁₋₄ alkyl groups, C₁₋₄ alkyl-(C₂₋₅ heterocycloalkyl) optionally substituted with 1 or 2 C₁₋₄ alkyl groups, C(O)—C₂₋₉ heteroaryl optionally substituted with 1 or 2 C₁₋₄ alkyl groups; SO₂-C₂₋₉ heteroaryl optionally substituted with 1 or 2 C₁₋₄ alkyl groups, or haloC₁₋₄ alkyl groups; or two adjacent RX groups together form C₁₋₃ alkylenedioxy;
RY is selected from H, hydroxyl, halo, C₁₋₄ alkyl, haloC₁₋₄alkyl, C₁₋₄ alkoxy.

In a further embodiment, the present invention relates to use of a heparanase inhibitor in the preparation of a medicament for the prevention or treatment of transplant rejection wherein said heparanase inhibitor inhibits heparanase activity and thereby prevents or treats transplant rejection and wherein said heparanase inhibitor is a compound of general Formula I, Formula II or Formula III, as described above, for example in paragraphs to above.

In another embodiment, the use of the invention comprises use of a heparanase inhibitor that is suitable for administration in combination with one or more immunosuppressive drugs. Suitable immunosuppressive drugs would be well known to the person of skill in the relevant art. Preferably, the one or more immunosuppressive drugs may be selected from the group consisting of methotrexate, mizoribine, cyclosporin, aerosolized cyclosporin, tacrolimus, mycophenolate mofetil, azathioprine, sirolimus and other mTOR inhibitors, deoxyspergualin, leflunomide, malononitriloamide analogs of leflunomide; anti-CTLA4 antibodies, anti-CTLA4 Ig fusions, anti-B lymphocyte stimulator antibodies, anti-CD80 antibodies, etanercept, infliximab, anti-T cell antibodies, anti-CD3 antibodies, OKT3, anti-CD4 antibodies, anti il-2 receptor antibodies, prednisolone or its derivatives, anti CD52 monoclonal antibodies; anti-CD20 monoclonal antibodies; belatacept; eculizumab; and intravenous immunoglobulin.

In a preferred embodiment, the immunosuppressive drug is cyclosporin A or tacrolimus.

In a further embodiment, the use of the invention comprises use of a heparanase inhibitor that is suitable for administration in combination with one or more anti-inflammatory drugs. Suitable anti-inflammatory drugs would be well known to the person of skill in the relevant art. Preferably, the one or more immunosuppressive drugs may be selected from the group consisting of corticosteroids, clobetasol, halobetasol, hydrocortisone, triamcinolone, betamethasone, fluocinolone, fluocinonide, prednisone, prednisolone and methylprednisolone.

In another embodiment, the present invention relates to the use of a heparanase inhibitor in the preparation of a medicament for the prevention or treatment of transplant rejection wherein said heparanase inhibitor is OGT 2115 having the formula

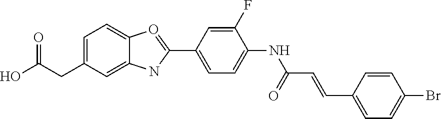

or a functional derivative or functional analog thereof, wherein said OGT 2115 inhibits heparanase and thereby prevents or treats transplant rejection.

In yet another embodiment, the present invention relates to the use of a heparanase inhibitor in the preparation of a medicament for the prevention or treatment of transplant rejection wherein said heparanase inhibitor is OGT 2115 having the formula

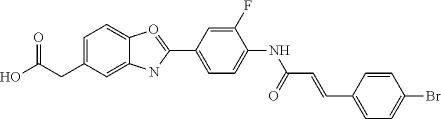

or a functional derivative or functional analog thereof, wherein said OGT 2115 inhibits heparanase activity and thereby prevents or treats transplant rejection and is suitable for administration in combination with cyclosporin A or tacrolimus.

Preferably, for the embodiments directed to the use of the invention, prevention or treatment of transplant rejection comprises prolonging the survival time of the transplant.

In yet a further aspect, the present invention relates to a heparanase inhibitor for use in the prevention or treatment of transplant rejection wherein said heparanase inhibitor inhibits heparanase activity and thereby prevents or treats transplant rejection.

In one embodiment, in relation to the heparanase inhibitor for use in the prevention or treatment of transplant rejection, the rejection is rejection of a primarily vascularized organ transplant. Preferably, the prevention or treatment treats or prevents transplant rejection of, for example but not limited to, a heart, heart-lung, kidney, lung, liver, pancreas, stomach or intestine transplant.

In a further embodiment, the transplant rejection is allograft transplant rejection.

In yet another embodiment, the heparanase inhibitor of the invention is a benzoxazol-5-yl acetic acid derivative. In a specific embodiment, the heparanase inhibitor is OGT 2115 having the formula,

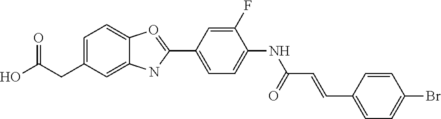

or a functional derivative or functional analog thereof.

In a further embodiment, the heparanase inhibitor used in the method of the invention is a quinazoline compound. For example, a compound of general formula A

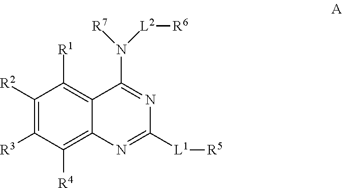

or a salt, hydrate, solvate, tautomer or stereoisomer thereof,
wherein:
R¹ is selected from H, hydroxyl, halo, C₁₋₆ alkyl, C₁₋₄ alkoxy, O-CH₂phenyl, O-phenyl;
R² is selected from H, hydroxyl, halo, C₁₋₆ alkyl, C₁₋₄ alkoxy, O-CH₂phenyl, O-phenyl;
R³ is selected from H, hydroxyl, halo, C₁₋₆ alkyl, C₁₋₄ alkoxy, O-CH₂phenyl, O-phenyl;
R⁴ is selected from H, hydroxyl, halo, C₁₋₆ alkyl, C₁₋₄ alkoxy, O-CH₂phenyl, O-phenyl;
or R¹ and R², or R² and R³, or R³ and R⁴ together form C₁₋₃alkylenedioxy;
wherein at least one of R¹, R², R³ and R⁴ is not H,
L¹ is selected from NHC₁₋₄alkyl-NHC(O)—, NHC₁₋₄alkyl-NHSO₂—, azetidinyl-NHC(O)—, and azetidinyl-NHSO₂—;
R⁵ is selected from C₃₋₉ cycloalkyl, C₆₋₁₀ aryl optionally substituted with 1 or 2 RX groups, C₂₋₉heteroaryl optionally substituted with 1 or 2 RX groups, C₂₋₅ heterocycloalkyl optionally substituted with 1 or 2 RX groups, C₁₋₄ alkylC₂₋₅ heterocycloalkyl optionally substituted with 1 or 2 RX groups;
L² is selected from C₁₋₄ alkyl, azetidinyl-C(O)—, C₁₋₄ alkyl-NHC(O)—, C₁₋₄ alkyl-NHSO₂—, —C(O)—, SO₂—; or L² is absent;
R⁶ is selected from H, C₂₋₆ alkyl, guanidinyl, NHC(NH)NH(C₁₋₃ alkyl), ureido, NHC(O)NH(C₁₋₃ alkyl), C₆₋₁₀ aryl optionally substituted with 1 or 2 RX groups, C₁₋₉ heteroaryl optionally substituted with 1 or 2 RX groups, C₂₋₅ heterocycloalkyl optionally substituted with 1 or 2 RX groups, C₃₋₉ cycloalkyl optionally substituted with 1 or 2 RX groups;
R⁷ is H or C₁₋₆ alkyl;
and wherein when L¹ is NHC₁₋₄alkyl-NHSO₂—, R⁵ is not phenyl substituted with one methyl, tert-butyl or phenyl group;
each RX is independently selected from hydroxyl, halo, nitro, NR′R″ (wherein R′ and R″ are independently selected from H and C₁₋₃ alkyl), C₁₋₄ alkyl, C₃-9 cycloalkyl, haloC₁₋₄ alkyl, C₁₋₄alkoxy, C(O)C₁₋₃ alkyl, C(O)OC₁₋₄ alkyl, C(O)NHRY, C₆₋₁₀ aryl optionally substituted with 1 or 2 RY groups, C₂₋₉ heteroaryl optionally substituted with 1 or 2 RY groups, C₁₋₄ alkyl-(C₂₋₉heteroaryl), C₂₋₅ heterocycloalkyl optionally substituted with 1 or 2 C₁₋₄ alkyl groups, C₁₋₄ alkyl-(C₂₋₅ heterocycloalkyl) optionally substituted with 1 or 2 C₁₋₄ alkyl groups, C(O)—C₂₋₉ heteroaryl optionally substituted with 1 or 2 C₁₋₄ alkyl groups; SO₂-C₂₋₉ heteroaryl optionally substituted with 1 or 2 C₁₋₄ alkyl groups, or haloC₁₋₄ alkyl groups; or two adjacent RX groups together form C₁₋₃ alkylenedioxy;
RY is selected from H, hydroxyl, halo, C₁₋₄ alkyl, haloC₁₋₄alkyl, C₁₋₄ alkoxy.

In a further embodiment, the present invention relates a heparanase inhibitor in the preparation of a medicament for the prevention or treatment of transplant rejection wherein said heparanase inhibitor inhibits heparanase activity and thereby prevents or treats transplant rejection and wherein said heparanase inhibitor is a compound of general Formula I, Formula II or Formula III, as described above, for example in paragraphs to above.

In a further embodiment, the heparanase inhibitor of the invention is administered in combination with one or more immunosuppressive drugs. For example, the immunosuppressive drug is selected from the group consisting of but not limited to, methotrexate, mizoribine, cyclosporin, aerosolized cyclosporin, tacrolimus, mycophenolate mofetil, azathioprine, sirolimus and other mTOR inhibitors, deoxyspergualin, leflunomide, malononitriloamide analogs of leflunomide; anti-CTLA4 antibodies, anti-CTLA4 Ig fusions, anti-B lymphocyte stimulator antibodies, anti-CD80 antibodies, etanercept, infliximab, anti-T cell antibodies, anti-CD3 antibodies, OKT3, anti-CD4 antibodies, anti il-2 receptor antibodies, prednisolone or its derivatives, anti CD52 monoclonal antibodies; anti-CD20 monoclonal antibodies; belatacept; eculizumab; and intravenous immunoglobulin.

In a certain embodiment, the heparanase inhibitor of the invention is administered in combination with cyclosporin A or tacrolimus.

In a further embodiment, the heparanase inhibitor of the invention is administered in combination with one or more anti-inflammatory drugs. For example, the anti-inflammatory drug is selected from the group consisting of but not limited to corticosteroids, clobetasol, halobetasol, hydrocortisone, triamcinolone, betamethasone, fluocinolone, fluocinonide, prednisone, prednisolone and methylprednisolone.

In yet a further embodiment, the heparanase inhibitor according to the invention prolongs the survival time of a transplant.

In a further embodiment, the heparanase inhibitor is administered in a dose from 1 mg/kg to 50 mg/kg. Specifically, the heparanase inhibitor is administered at a dosage of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg. In a further embodiment the heparanase inhibitor is administered at a dosage of 2.0 mg/kg to 10 mg/kg. Specifically, this includes 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0, 6.5 mg/kg, 7.0 mg/kg, 7.5 mg/kg, 8.0 mg/kg, 8.5 mg/kg, 9.0 mg/kg, 9.5 mg/kg, or 10 mg/kg.

The heparanase inhibitor may be administered to a subject at any one of the dosages described above daily, for example once a day. Alternatively, the heparanase inhibitor is administered weekly, for example once weekly.

In a further aspect, the present invention relates to a heparanase inhibitor for use in the prevention or treatment of transplant rejection wherein said heparanase inhibitor inhibits heparanase activity and thereby prevents or treats transplant rejection.

It would be well known to the skilled person that the heparanase inhibitors of the invention can be administered by any suitable dosage route, for example by parenteral administration, including but not limited to subcutaneous administration, intravenous administration, intradermal administration, intramuscular administration, and intraperitoneal administration. The skilled person would also understand that the heparanase inhibitors of the invention can be administered by non-parenteral administration, for example orally, or rectally.

It would be clear to the person of skill in the art that the present invention is not limited to the use of OGT 2115 and that other specific heparanase inhibitors, for example those described above, would be suitable in the method and use of the invention to treat transplant rejection, including prolonging the survival of transplants for, but not limited to, allograft organ transplants.

In particular, it would be a matter of routine for the skilled person to identify heparanase inhibitors suitable for the methods and uses of the present invention. Suitable assays for determining heparanase inhibitory activity are known in the art, for example, the in vitro assays described in Rivara et al. (2016) Future Med Chem, 8(6): 647-680. For example, the method may include contacting a preparation comprising heparanase and a heparanase substrate (e.g., heparan sulphate or fondaparinux) with a test compound and detecting the amount of the intact substrate in comparison to a reference level of intact substrate in the absence of the test compound or detecting the modulation of the activity of a downstream target of the intact heparanase substrate. Detecting the amount of intact substrate or modulation may be achieved using techniques including, but not limited to, ELISA, cell-based ELISA, inhibition ELISA, western blots, RIA, immunoprecipitation, immunostaining, a solid-phase labelled substrate assay such as a solid phase radio- or fluorescently-labelled or biotinylated substrate, an ultrafiltration assay, proximity assays such as HTRF and scintillation proximity assays, fluorescent assays using e.g., fluorescent substrate-heparanase substrate conjugates such as fluorescein or rhodamine, colorimetric assays and fluorescent immunoassays, all of which are well known to those skilled in the art.

The skilled person could readily prepare, for example, a derivative or analog of OGT 2115 or compound of Formula A, Formula I, Formula II, or Formula III using well known chemical synthesis and test for heparanase inhibitory activity using an assay, for example, as described in paragraph above.

Other and further aspects and features of the disclosure will be evident from reading the following detailed description of the embodiments, which are intended to illustrate, not limit, the present disclosure.

### BRIEF DESCRIPTION OF THE FIGURES

The illustrated embodiments of the disclosed subject matter will be best understood by reference to the Figures and Tables, wherein like parts are designated by like numerals throughout. The following description is intended only by way of example, and simply illustrates certain selected embodiments of devices, systems, and processes that are consistent with the disclosed subject matter as claimed herein.

FIG. 1 shows the distribution of rat heart allograft survival times in days. The black dots within the boxplots correspond to the raw data points of Table 2. Each black dot represents the survival of one rat that had a heterotopically transplanted heart.

FIG. 2 is a diagrammatic representation of the percentage abundance of cDNA for each of the experimental groups in 2A) kidney and 2B) blood samples described in Example 2.

### DETAILED DESCRIPTION

Preferred features, embodiments and variations of the invention may be discerned from the following detailed description which provides sufficient information for those skilled in the art to perform the invention. The detailed description is not to be regarded as limiting the scope of the preceding summary of the invention in any way.

### Definitions

In the context of the present invention, the words “comprise”, “comprising” and the like are to be construed in their inclusive, as opposed to their exclusive, sense, that is in the sense of “including, but not limited to”.

Throughout the specification and claims (if present), unless the context requires otherwise, the term “substantially” or “about” will be understood to not be limited to the value for the range qualified by the terms. Accordingly, unless indicated to the contrary, the numerical parameters set forth in the following specification and attached claims are approximations that may vary depending upon the desired properties sought to be obtained by the present invention. At the very least, each numerical parameter should be construed in light of the number of significant digits and ordinary rounding conventions. The term “about” may be understood to refer to a range of +/−10%, such as +/−5% or +/−1% or, +/−0.1%.

The terms “a,” “an,” “the” and similar referents used in the context of describing the invention (especially in the context of the following claims) are to be construed to cover both the singular and the plural, unless otherwise indicated herein or clearly contradicted by context.

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention otherwise claimed. No language in the specification should be construed as indicating any non-claimed element is essential to the practice of the invention, unless otherwise indicated herein or clearly contradicted by context.

In the context of the present invention, the term “transplantation” insofar as it relates to tissues and organs may include an “autograft transplant,” “allograft transplant” or “xenograft transplant”. These terms are further defined as follows.

The term “autograft transplant” in the context of the present invention is defined as transplantation of cells, tissues, or organs between sites within the same individual or into an identical twin.

The terms “allograft”, “allograft transplant” or “allotransplant” in the context of the present invention is defined as transplantation of organs or tissues from a donor to a non-genetically identical individual of the same species.

The term “xenograft transplant” in the context of the present invention is defined as transplantation of an organ or tissue between two different species.

In the context of the above defined transplants, the term “primarily vascularized organ transplant” (PVT) refers to an organ transplant that is primarily vascularised, such as but not limited to a heart, heart-lung, kidney, lung, and liver transplant. Primarily vascularized transplants are organs that are connected to the recipient's blood supply by surgical arterial and venous anastomoses (linkages) and gain an immediate blood supply.

In the context of the above defined transplants, the term “secondarily vascularized transplant” (SVT) refers to a tissue transplant that is secondarily vascularised such as, but not limited to, an islet cell, skin or cornea transplant. Secondarily vascularized transplants are cellular or tissue transplants that do not have vascular anastomoses but are usually lodged into another organ or under its capsule using catheters lodged into the recipient's veins. To survive, SVTs need to grow a network of capillaries that link into the blood supply of the recipient. This may take days to weeks. PVTs are subject to immediate antibody attack from recipient alloantibodies that can be generated from pregnancy, pregraft blood transfusion or previous allotransplants. Such an attack usually causes allograft loss often on the operating table. SVTs do not have their blood supply created surgically but require the slow growth of blood vessels and are almost never subject to this attack. PVTs gain immediate function more quickly than SVTs. SVTs usually have relatively long ischaemia times (time between procurement from donor and implantation/revascularization in the transplant recipient) compared with PVTs. For example, islets are removed from the pancreas by chemical digestion of the pancreas after its procurement from the deceased donor, perfused, washed and stored at 4° C., tested functionally and then transplanted—at least 24/24. By contrast, for live donor renal transplantation the warm ischaemia time is 2-3 minutes and cold ischaemia time about 1-2 hours. Hence studies on islet cell survival need to have controls for ischaemia reperfusion injury.

For example, a skin tissue allotransplant is secondarily vascularized and therefore not likely to be subject to immediate vascular rejection by alloantibodies. By contrast, a heart allotransplant is primarily vascularized and therefore subject to immediate alloantibody attack.

The term “inhibitor” as used herein refers to an agent that decreases, inhibits, or impairs at least one function or biological activity of a target molecule.

As used herein, the term “heparanase inhibitor” refers to an agent that decreases, inhibits or impairs at least one function or biological activity of heparanase. The term “heparanase inhibitor” includes an agent that decreases the biological activity of heparanase. The term “heparanase inhibitor” also includes an agent that inhibits or reduces heparanase activity by reducing the level of heparanase, for example, the expression levels of heparanase. The person skilled in the art would understand, for example, that a reduction in expression levels of heparanase would result in a reduction in heparanase activity.

In the context of the present invention, the properties of a heparanase inhibitor include a capacity to treat allotransplant rejection by, for example, prolonging the survival of an allotransplant in vivo.

The term “transplant site” refers to the site of the transplanted tissue or transplanted organ. It also refers to the transplanted tissue or the transplanted organ. In this regard, the phrase “lymphocytes at a transplant site” refers to the lymphocytes within and in the vicinity of the transplanted tissue or transplanted organ.

In the context of the present invention, the phrase “heparanase inhibitor reduces expression levels of heparanase”, and the like, means a lowering of gene heparanase expression levels, for example measured as a reduction in heparanase mRNA or corresponding cDNA in subjects treated with heparanase inhibitor as compared with gene heparanase expression levels in subjects not treated with heparanase inhibitor.

The term “subject” includes any human or non-human mammal. Thus, in addition to being useful for human treatment, the compounds of the present invention may also be useful for veterinary treatment of mammals, including companion animals and farm animals, such as, but not limited to dogs, cats, horses, cows, sheep, and pigs. In preferred embodiments the subject is a human.

The term “therapeutically effective dose” refers to an amount of an agent sufficient to produce a desired therapeutic or pharmacological effect in the subject being treated. The term is synonymous and intended to qualify the amount of agent that will achieve the goal of improvement in disease severity and/or the frequency of incidence over treatment of each agent by itself while preferably avoiding or minimising adverse side effects, including side effects typically associated with other therapies. It would be a matter of routine for a skilled person to determine a therapeutically effective dose using information and routine methods known in the art.

The compounds as used in the methods of the invention described herein may be administered as a formulation comprising a pharmaceutically effective amount of the compound, in association with one or more pharmaceutically acceptable excipients including carriers, vehicles and diluents. The term “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solid dosage form such as a tablet, capsule, or a solution or suspension suitable for oral, parenteral, intradermal, or subcutaneous administration. Excipients can include, by way of illustration and not limitation, diluents, disintegrants, binding agents, adhesives, wetting agents, polymers, lubricants, glidants, stabilizers. Acceptable excipients include (but are not limited to) stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutically acceptable materials. Examples of excipients and their use is described in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

The term “pharmaceutically acceptable salt” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19.

The compounds and pharmaceutical compositions as used in the method of the invention may be formulated for oral, injectable, rectal, parenteral, subcutaneous, intravenous, topical, intravitreal or intramuscular delivery. Non-limiting examples of particular formulation types include tablets, capsules, caplets, powders, granules, injectables, ampoules, vials, ready-to-use solutions or suspensions, lyophilized materials, suppositories and implants. Solid formulations such as the tablets or capsules may contain any number of suitable pharmaceutically acceptable excipients or carriers described herein. The compounds of the invention may also be formulated for sustained delivery.

Tablets and capsules for oral administration may be in unit dose presentation form, and may contain conventional excipients such as binding agents, for example, acacia, gelatin, sorbitol, tragacanth, or polyvinylpyrrolidone; fillers, for example lactose, sugar, maize-starch, calcium phosphate, sorbitol or glycine; tabletting lubricants, for example, magnesium stearate, talc, polyethylene glycol or silica; disintegrants, for example, potato starch; or acceptable wetting agents such as sodium lauryl sulphate. The tablets may be coated according to methods well known in normal pharmaceutical practice.

For parenteral administration, including intravenous, intramuscular, subcutaneous, or intraperitoneal administration, fluid unit dosage forms may be prepared by combining the compound and a sterile vehicle, typically a sterile aqueous solution which is preferably isotonic with the blood of the recipient. Depending on the vehicle and concentration used, the compound may be either suspended or dissolved in the vehicle or other suitable solvent. In preparing solutions, the compound may be dissolved in water for injection and filter sterilized before filling into a suitable vial or ampoule and sealing. Advantageously, agents such as a local anaesthetic, preservative and buffering agents can be dissolved in the vehicle. To enhance the stability, the composition may be frozen after filling into the vial and the water removed under vacuum. The dry lyophilized powder may then be sealed in the vial and an accompanying vial of water for injection or other suitable liquid may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. A surfactant or wetting agent may be included in the composition to facilitate uniform distribution of the compound.

In certain embodiments the compounds as used in the method of the invention are formulated as an injectable solution, suspension or emulsion.

A “pharmaceutical carrier, diluent or excipient” includes, but is not limited to, any physiological buffered (i.e., about pH 7.0 to 7.4) medium comprising a suitable water-soluble organic carrier, conventional solvents, dispersion media, fillers, solid carriers, coatings, antibacterial and antifungal agents, isotonic and absorption delaying agents. Suitable water-soluble organic carriers include, but are not limited to, saline, dextrose, corn oil, dimethylsulphoxide, and gelatin capsules. Other conventional additives include lactose, mannitol, corn starch, potato starch, binders such as microcrystalline cellulose, cellulose derivatives such as hydroxypropylmethylcellulose, acacia, gelatins, disintegrators such as sodium carboxymethylcellulose, and lubricants such as talc or magnesium stearate.

The terms “therapeutically effective amount” or “pharmacologically effective amount” or “effective amount” refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimising adverse side effects, including side effects typically associated with other therapies. Those skilled in the art can determine an effective dose using information and routine methods known in the art.

It would also be known to the skilled person that there are further differences between a secondarily vascularized transplant and a primarily vascularized transplant. For example, cell trafficking and the basement membrane into the allotransplant and that these differences may affect how the rejection occurs and, also how the rejection may be treated.

In the context of the present invention the term “in combination with” insofar as it relates to administration of compositions in combination with each other, means that the compositions may be administered together, for example at the same time, or sequentially in any order and given over a period of time that results in the two compositions acting together to treat transplant rejection.

In the context of the present invention the term “functional derivative or functional analog” insofar as it relates to a specific compound, means a compound that is structurally similar to the specific compound and exhibits the same functional properties as the specific compound. For example, a functional derivative or functional analog of OGT 2115 includes compound that is a benzoxazol-5-yl acetic acid derivative, and includes a salt, hydrate, solvate, tautomer or stereoisomer thereof of a relevant compound, which is capable of inhibiting heparanase activity.

Any embodiment of the invention is meant to be illustrative only and is not meant to be limiting to the invention. Therefore, it should be appreciated that various other changes and modifications can be made to any embodiment described without departing from the spirit and scope of the invention.

As used herein, “treat”, “treating” or “treatment” of allotransplant rejection means accomplishing one or more of the following: (a) reducing the severity and/or duration of the rejection; (b) ceasing rejection; (c) limiting or preventing development of symptoms characteristic of the rejection being treated; (d) inhibiting worsening of symptoms characteristic of the rejection being treated; (e) limiting or preventing recurrence of the rejection in patients that have previously shown signs of rejection; and (f) limiting or preventing recurrence of symptoms in patients that were previously symptomatic for rejection. The terms “treat”, “treating” or “treatment” do not necessarily imply that a subject is treated until total recovery.

As used herein, “prevent”, “preventing”, “prevention”, or “prophylaxis” of allotransplant rejection means preventing allotransplant rejection in a subject.

As used herein, “maintaining the integrity” of an allotransplant, or “maintaining” an allotransplant, in a subject means keeping the allotransplant substantially healthy, functional and keeping the allotransplant's biological structure substantially intact.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

A preferred embodiment of the invention is further described by the following non-limiting examples with reference to the accompanying figures.

### EXAMPLES

### Materials and Methods

Inbred rat strains PVG (RT1c) (donor) and DA (RT1a) (recipient) used for heart and renal transplantation were housed under standard conditions. All procedures were approved and audited by an Animal Care and Ethics Committee in Australia.

### Rat Heterotopic Cardiac Transplantation

Heterotopic cardiac transplants were done using a standard technique (10). Cardiac function was assessed daily by abdominal palpation. The end point of cardiac transplant survival was defined as the last day of palpable heart beating. Care of all rats in this study complied with the Animal Research Act 1985 (NSW, Australia). The protocols were designed to minimise pain and discomfort to the animals. Animals were acclimatised to laboratory conditions (22° C., 12 h cycle of light and dark, 50% humidity, ad libitum access to food and water) for a minimum of 1 wk. prior to experimentation. Intragastric gavage administration was carried out with conscious animals, using curved gavage needles appropriate for animal size (250-300 gm body weight: Gauge 16, 100 mm). All transplanted rats were given post-operative analgesia (Carprofen 4 mg/kg every 12-24 h subcutaneously). They were euthanized by approved carbon dioxide asphyxiation when the heart stopped beating prior to tissue procurement.

### Rat Renal Transplantation

Left orthotopic renal transplants were performed with a mean anastomosis time of 37±4 minutes using a standard technique (11), which would be well known to the skilled person. The procured kidney was perfused with 2 ml Custodial HTK solution (Chemie GMBH, Alsbach-Hähnlein, Germany) at 40 C. Transplanted rats were given analgesia (Carprofen 5 mg/kg every 12-24 hours subcutaneously) and a prophylactic antibiotic (Enrofloxacin 5 mg/kg SC 12 hourly) for 3 days. The transplanted kidney and blood were procured on day 6 immediately after euthanasia by CO₂ asphyxiation. The kidney and blood were used for measurements of gene expression in intragraft lymphoid cells and peripheral blood mononuclear cells.

### Study Design

Studies on heart allograft survival were performed on six Groups: Isograft (control for ischaemia reperfusion injury); Non-treated (Rejecting) Allograft; Dimethyl Sulphoxide (DMSO) (control for the carrier for OGT 2115); OGT 2115 (heparanase inhibitor)-treated Allografts with 3 doses: 2.5 mg/kg; 5 mg/kg; and 10 mg/kg. Studies on gene expression were performed on five Groups: Isograft (control for ischaemia reperfusion injury); Non-treated (Rejecting) Allograft; OGT 2115 (heparanase inhibitor)-treated Allograft; and Castanospermine-treated Allograft. For these studies OGT 2115 was administered at 10 mg/kg. Studies about gene expression of heparanase were performed on lymphoid cells from transplanted kidney tissue and peripheral blood on day 6 after transplantation. This time frame encompassed the period of maximum rejection at day 6.

### Drug Administration

The Heparanase Inhibitor (OGT 2115) was given subcutaneously at 2.5 mg/kg or 5 mg/kg or 10 mg/kg after it was suspended in dimethyl sulphoxide and saline. Castanospermine was administered by Alzet osmotic pumps (Alza Corporation, Palo Alto, United States) at an immunosuppressive dose of 150 mg/kg per day. OGT 2115 treatment started on the first post operative day and the Castanospermine treatment started on the day of the transplant.

### Cell Procurement

Briefly, kidney tissue was minced and digested with 200 unit/ml of collagenase type 4 (Worthington Biochemical Corp, Lakewood, New Jersey, USA). Tissue suspension was passed through a sieve followed by a 70 μm nylon cell strainer (Falcon Corning Inc, Corning, NY USA). Lymphoid cells from peripheral blood were prepared using a standard method. 100 μl aliquots (1×10⁶ cells) of the resulting cell suspensions for kidney and blood were then made.

### Samples

Cell suspensions (1×10⁶ cells) of tissue and blood were suspended in RNAlater™ Stabilization Solution (Sigma, St Louis, MO, USA) and frozen at −80° C.

### Isolation of RNA

Cell suspensions were thawed prior to use. 5 ml cold PBS was added and cells were resuspended before being spun at 6000 g for 10 minutes. The supernatant was removed and 600 ul RLT Plus buffer (Qiagen, Hilden, Germany) was added and samples were resuspended before vortexing to ensure complete homogenisation of the sample.

RNA was manually extracted using the RNeasy Plus Mini Kit as per the manufacturer's instructions (Qiagen, Hilden, Germany). RNA was eluted in 30 ul RNase-free water. The quality of the RNA was measured using the Nanodrop 2000 (ThermoFisher Scientific, MA, USA) and quantified using the Qubit 2.0 Fluorometer (Invitrogen, MA, USA), RNA was stored at −800 C. Conversion of RNA to cDNA was performed using the iScript Advanced cDNA Synthesis Kit for RT-qPCR (Bio-Rad, Hercules, CA, USA) as per the manufacturer's instructions. cDNA samples were stored at −200 C.

Droplet Digital PCR (ddPCR)

Custom ddPCR assays were designed using the IDT PrimerQuest™ Tool, Prime Time qPCR assay (IDT, Singapore). The probes were tagged with either reporter dye 5′-FAM™ or 5′-HEX™ and with the fluorescent quencher 3′ lowa Black®. The ddPCR assays were resuspended in IDTE and diluted to a working concentration of 900 nM primers/250 nM probe. Primer and probe sequences for each assay can be found in paragraph Error! Reference source not found. below. A 25 ul reaction containing, ddPCR SuperMix for Probes (No dUTP) (Bio-Rad, Hercules, CA, USA), a hypoxanthine-guanine phosphoribosyl transferase reference control (HPRT-1) and heparanase target probes (HPSE) (IDT, Singapore). cDNA and water were created for each sample and added to a 96 well plate ready for ddPCR droplet generation.

ddPCR was performed using the QX200 AutoDG Droplet Digital PCR System (Bio-Rad, Hercules, CA, USA). After droplet generation, samples were placed on a thermocycler with the following conditions:

| Cycling | Temperature | | | Ramp | Number |
|---|---|---|---|---|---|
| Step | (° C.) | Time | | Rate | of Cycles |
| Enzyme Activation | 95 | 10 | min | 1° C./sec | 1 |
| Denaturation | 94 | 30 | sec | | 40 |
| Annealing/Extension | 56 | 2 | min | | 40 |
| Enzyme Deactivation | 98 | 10 | min | | 1 |

| | | | |
|---|---|---|---|
| Hold (optional) | 4 | Infinite | 1 |

The samples were left on the cycler for a minimum of 4 hours at 4° C. The droplets were then read on the QX200 Droplet Reader and analysed using QuantaSoft Software (Bio-Rad, Hercules, CA, USA). Results were expressed using the parameter fractional abundance of cDNA.

### HPSE Primer/Probe Assay

| Contains: | nmoles | Sequence |
|---|---|---|
| Probe | 2.5 | 5′-/56-FAM/CATCAGCAG/ZEN/ |
| | | GACACGTCATCAT/3IAkKFQ/-3′ |
| Primer 1 | 9.0 | 5′-CAGAGAGGGAGAAAGAAGGTAG-3′ |
| Primer 2 | 9.0 | 5′-CATAAGTTGTTGAAGTGAGGTAGAG-3′ |

HPSE Primer 1 sequence above is Seq ID NO. 1.
HPSE Primer 2 sequence above is Seq ID NO. 2.
HPSE Probe sequence above is Seq ID NO. 3 with FAM fluorophore and 3IAbkFQ and ZEN fluorescent quenchers. The skilled person would understand that these are required to ensure that the dyes can be read accurately.

### HPRT1 Primer/Probe Assay

| Contains: | nmoles | Sequence |
|---|---|---|
| Probe | 2.5 | 5′-/SHEX/CGTCTGCTA/ZEN/ |
| | | GTTCTTTACTGGCC/3IABkFQ/-3′ |
| Primer 1 | 9.0 | 5′-TAGATGGCCACAGGACTAGA-3′ |
| Primer 2 | 9.0 | 5′-TGAGCACTTCAGGGATTTGA-3′ |

HPRT1 Primer 1 sequence above is Seq ID NO. 4.
HPRT1 Primer 2 sequence above is Seq ID NO. 5
HPRT1 Probe sequence above is Seq ID NO. 6 with HEX fluorophore and
3IAbkFQ and ZEN fluorescent quenchers. The skilled person would understand that these are required to ensure that the dyes can be read accurately.

### Example 1

An experiment was conducted comparing heart allograft survival in 6 groups of inbred rats. Three of these groups, 4, 5 and 6 were treated with the specific heparanase inhibitor OGT 2115 The groups are set out in Table 1 as follows:

TABLE 1

| Group | Name |
|---|---|
| 1 | Isograft DA-DA |
| 2 | Non-treated PVG-DA |
| 3 | DMSO |
| 4 | OGT 2115 2.5 mg/kg |
| 5 | OGT 2115 5 mg/kg |
| 6 | OGT 2115 10 mg/kg |

The data from the first group of rats is not included in these analyses, leaving 5 groups of interest. All 11 rats transplanted with isografts survived to 100 days before being euthanized.

There are 2 co-primary sets of null hypotheses:

$$\begin{matrix}1. & \\ M 2 = M 4 ; M 2 = M 5 ; M 2 = M 6 & \end{matrix}$$

$$\begin{matrix}2. & \\ M 3 = M 4 ; M 3 = M 5 ; M 3 = M 6 & \end{matrix}$$

And 2 secondary null hypotheses:

$$\begin{matrix}3. & \\ M 2 = ( M 4 + M 5 + M 6 ) / 3 & \end{matrix}$$

$$\begin{matrix}4. & \\ M 3 = ( M 4 + M 5 + M 6 ) / 3 & \end{matrix}$$

These tests were assessed using the Wilcoxon rank sum test procedure. The mean, median, min, max and the first and third quartiles are presented for each group, as well as the estimated difference in location parameters with 95% confidence intervals. The difference in location parameters is the median of the difference between a sample from the first group and a sample from the second group.

The study was powered such that a sample of 11 rats in each of groups 2, 3 and 6 would give the study 80% power to reject the null hypothesis with a type 1 error rate of 5% and detect an alternative hypothesis that the survival times are 20% greater in group 6 compared to group 2 and group 3.

TABLE 2
Summaries of the heart allograft survival
times (days) in each experimental group

| Group | N | mean | Min | q1 | median | Q3 | Max |
|---|---|---|---|---|---|---|---|
| 2 | 11 | 6.91 | 6 | 6.0 | 7 | 7.00 | 9 |
| 3 | 11 | 6.18 | 6 | 6.0 | 6 | 6.00 | 7 |
| 4 | 5 | 7.40 | 6 | 7.0 | 8 | 8.00 | 8 |
| 5 | 6 | 7.33 | 7 | 7.0 | 7 | 7.75 | 8 |
| 6 | 11 | 9.18 | 7 | 8.5 | 9 | 10.00 | 11 |

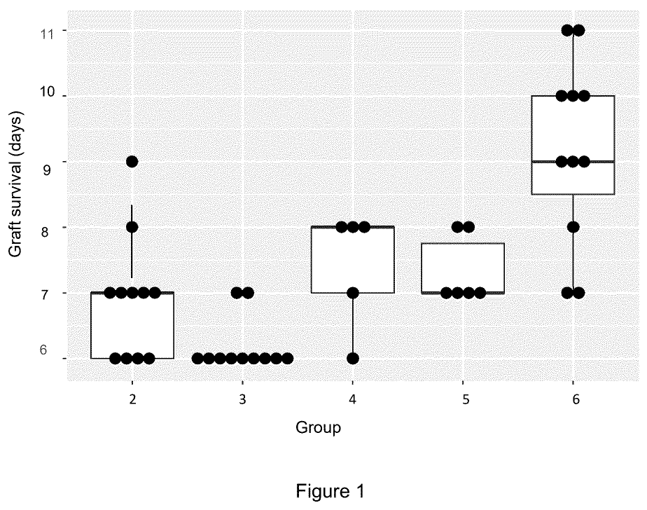

Table 2 summarises the heart allograft survival times in days for the five groups of rats. A plot of the distribution of the survival times for each group is shown in FIG. 1. The results shown in Table 2 and FIG. 1 display a clear trend that administration of OGT 2115 prolonged the survival of the heart allotransplant grafts. Specifically, the mean and median survival of the heart allotransplant grafts were increased in the lower dose groups (2.5 mg/Kg and 5 mg/Kg) and significantly increased in the highest dosage group (10 mg/Kg).

### Conclusions

The experimental treatment (use of an heparanase inhibitor) resulted in a significantly higher distribution of allograft survival days compared with the non-treated control group or compared with the DMSO control group. The highest dosage group (Group 6: 10 mg/Kg) had the greatest difference compared with the control groups (estimated increase in survival of 2 days compared to Non-treated PVG-DA and 3 days compared to DMSO). There was also a clear trend that mean and median transplant survival were increased in the lower dose groups when compared to both controls.

### Example 2

Heparanase expression levels were measured in lymphoid cells extracted from inbred rat kidney transplants at day 6 after transplantation. Heparanase expression levels were also measured in peripheral blood lymphoid cells isolated from the same rats. The heparanase levels were measured by isolating mRNA which was converted to cDNA by reverse transcriptase. The cDNA is measured using the parameter of fractional abundance. The experimental design consisted of 4 treatment groups, namely a Castanospermine (Cast) treatment group, a OGT 2115 treatment group, an isograft treatment group and a non-treated control group. All experimental groups had 3 rats and each rat had a maximum of 3 observations per rat. For the kidney group there were 21 observations in total (Table 3). For the blood group there were 24 observations in total. Two of the assays had experimental duplicates, these results were averaged as part of the data preparation.

TABLE 3
Number of rats with measured percent DNA from the kidney
samples in each experimental group, each rat had a
maximum of 3 assays under their given treatment

| | |
|---|---|
| Experimental | Assay |

| group | 1 | 2 | 3 | Total |
|---|---|---|---|---|
| Cast 150 mg/kg | 0 | 3 | 3 | 6 |
| Isograft | 0 | 0 | 3 | 3 |
| Non-Treated | 3 | 0 | 3 | 6 |
| OGT 2115 10 mg/kg | 0 | 3 | 3 | 6 |
| Total | 3 | 9 | 12 | 21 |

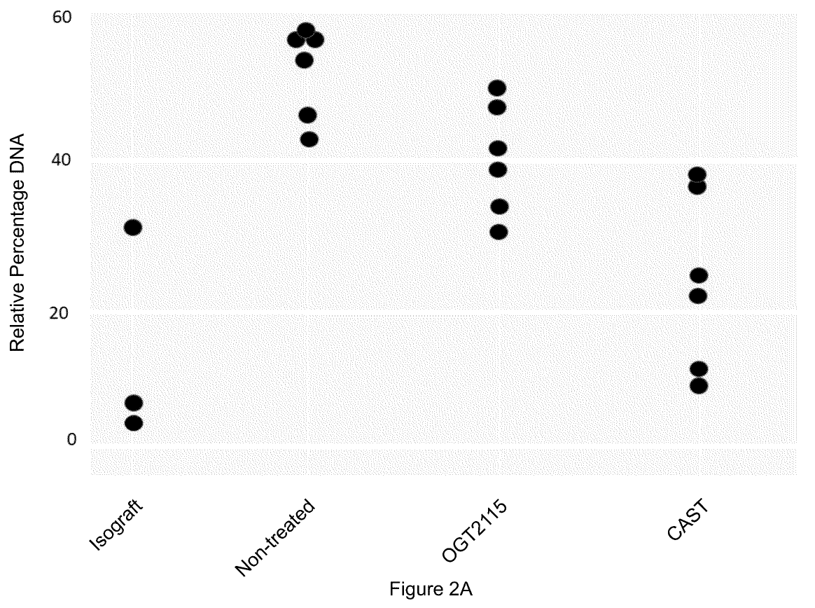

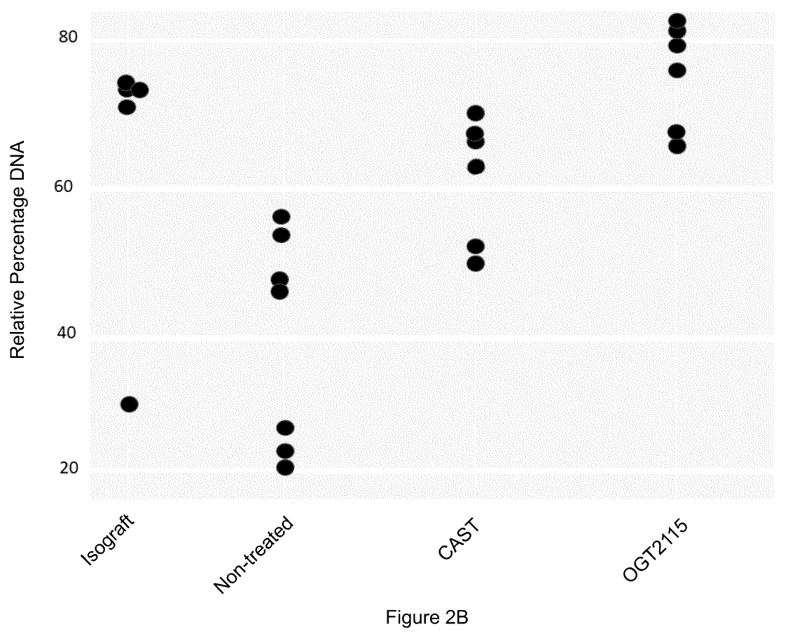

FIG. 2A is a diagrammatic representation of the percentage abundance of cDNA in lymphoid cells extracted from inbred rat kidney transplants, whereas FIG. 2B is a representation of the percentage abundance of cDNA in peripheral blood lymphoid cells isolated from the same rats.

### Statistical Methods

The contrasts specified in Table 4 were estimated using linear mixed effects regression models, with separate models estimated for the relative percentage of DNA in the kidney lymphoid cell data and the blood. The model included fixed categorical effects for the experimental groups (non-treated as the reference group), and a random subject specific intercept to account for the repeated measures from up to 3 assays per rat. Duplicate measures were averaged within the rat/assay prior to analysis. The residuals from this model were assumed to be from a Normal distribution, which was assessed using Normal quantile plots, and the variance of these residuals were initially assumed to be constant (assessed through inspecting plots of standardised residuals against fitted values). This assumption was latter relaxed to allow the variance to vary within the treatment groups. Contrast estimates are presented together with 95% confidence intervals, with degrees of freedom estimated using the Satterthwaite approximation. All analyses were performed in the R programming environment (R version 4.2.0) and linear mixed models estimated using the ‘Ime’ function from the ‘nlme’ package.

### Results

The mean differences in percentage of heparanase expression levels (heparanase cDNA levels) for lymphocytes isolated from kidney transplants are detailed in Table 4. Table 4 shows clear differences in heparanase levels between the heparanase inhibitor OGT 2115 and non-treated groups.

Specifically, the percent difference in heparanase expression levels in transplant isolated lymphocytes from the OGT 2115 (10 mg/kg) compared with non-treated transplant rats was-14.5%, indicating the OGT 2115 group had 14.5% lower heparanase expression levels than the non-treated group. Further, the difference in heparanase expression levels in transplant isolated lymphocytes from the Castanospermine-treated transplants compared with non-treated transplant rats was-31%. Thus, the Castanospermine-treated group had 31% lower heparanase expression levels compared with the non-treated group. Therefore, in rats treated with heparanase inhibitor OGT 2115, heparanase expression levels were clearly reduced in transplant lymphocytes compared with the non-treated group. Without wishing to be bound by theory, the results in Table 4 indicate that systemic administration of heparanase inhibitors specifically reduced heparanase expression levels in transplant lymphocytes involved in an allograft immune rejection response.

TABLE 4
Estimated mean differences in heparanase levels (percent heparanase
cDNA) for the kidney samples (n = 12 rats; 21 observations)

| Contrast | estimate | SE | df | lower.CL | upper.CL | t.ratio |
|---|---|---|---|---|---|---|
| (OGT 2115 10 mg/kg) - (Non-Treated) | −14.5 | 10.4 | 8.06 | −38.4 | 9.38 | −1.40 |
| (Cast 150 mg/kg) - (Non-Treated) | −31.0 | 10.4 | 8.02 | −54.8 | −7.12 | −2.99 |
| (OGT 2115 10 mg/kg) - Isograft | 26.4 | 10.4 | 8.03 | 2.5 | 50.23 | 2.55 |

To determine whether systemic administration of heparanase inhibitors produced an expected general reduction of heparanase in all lymphocytes, heparanase expression levels were determined for lymphocytes isolated from the peripheral blood of treated and untreated transplanted rats. These results are set out in Table 5. Unexpectedly, the level of heparanase in peripheral blood lymphocytes isolated from rats treated with heparanase inhibitors were not reduced compared with non-treated rats.

TABLE 5
Estimated mean differences in heparanase levels (percent heparanase
cDNA) for peripheral blood samples (n = 12 rats; 24 observations)

| Contrast | estimate | SE | df | lower.CL | upper.CL | t.ratio |
|---|---|---|---|---|---|---|
| (OGT 2115 10 mg/kg) - (Non-Treated) | 35.9 | 12.9 | 8.01 | 6.04 | 65.7 | 2.77 |
| (Cast 150 mg/kg) - (Non-Treated) | 22.1 | 13.0 | 8.05 | −7.75 | 51.9 | 1.71 |
| (OGT 2115 10 mg/kg) - Isograft | 17.0 | 12.9 | 7.98 | −12.80 | 46.8 | 1.32 |

### Conclusions

The results of Example 2 clearly show that systemic administration of heparanase inhibitors reduced the expression level of heparanase in transplant isolated lymphocytes. However, this reduction in heparanase expression levels was not observed in lymphocytes isolated from the peripheral blood of heparanase inhibitor treated transplant rats. Thus, the results indicate that the systemic administration of heparanase inhibitors to transplant recipients reduced the expression of heparanase in a targeted manner at the site of an immune rejection response.

Heparanase is known to be a complex multi-functional enzyme involved in many systems and processes in the body. The inventors have shown, in Example 1, that administration of heparanase inhibitors prolonged allotransplant survival times. The results of Example 2 reveal an unexpected and surprising benefit that the treatment of transplant rejection by heparanase inhibitors is specifically targeted to reducing heparanase expression levels at the site of the transplant. For this reason, the treatment of transplant rejection by heparanase inhibitors is unlikely to affect the wide spectrum of biological systems and processes in which heparanase may play a role, other than an immune rejection response at the site of a transplant. It follows that the targeted treatment of transplant rejection by administration of heparanase inhibitors is, for example, unlikely to have significant side effects and therefore be beneficial as a treatment for transplant rejection.

### Example 3

Experiments were conducted to determine the impact of Castanospermine treatment of renal allotransplanted rats. Specifically, the inventor understood that the integrity of cell membranes, extracellular matrix and vascular endothelium basement membrane are dependent on heparan sulphate proteoglycan (HSPG). The inventor hypothesised that, if HSPG is broken down by heparanase, this would allow alloreactive cell entry to allotransplants. As a consequence, heparan sulphate moieties containing inflammatory cytokines and chemokines would be released from HSPG, thus activating alloreactive cells and rejection. Therefore, it was of interest to determine Castanospermine's ability to stop the breakdown of HSPG entities and hence conserve allotransplant organ integrity.

### Groups and Statistical Analysis

Groups used in this study are set out in Tables 6 and 7. Comparisons were made between an isograft control treatment group (i.e., a control for ischaemia reperfusion injury), a non-treated allograft group, and Castanospermine-treated allograft group. A primary comparison was made between the non-treated group versus the Castanospermine-treated group. Secondary comparisons were made between the non-treated group versus the isograft group; and Castanospermine-treated group versus the isograft group.

Because multiple post-hoc comparisons were done on each of the data sources, type I errors were possible. Multiple methods were used to address this:
1. raw p values using Kenwood-Roger approximations for the degrees of freedom and comparisons with a Bonferroni corrected threshold;
2. adjusted p values using Benjamini and Hochberg procedure to control the false discovery rate to 5%; and
3. Bayes Factors to measure how much evidence the data provides in support (or against) the null hypothesis. Bayes Factors can be interpreted using the scale of Kass and Raftery (Journal of the American Statistical Association Vol. 90, No. 430:773-795). Under this analysis, values between 1 and 3.2 are not relevant; 3.2 to 10 are considered substantial; 10 to 100 strong; and, greater than 100 are considered decisive.

Adjustments were made for multiple testing within the grouping of hypotheses associated with each of the primary and secondary aims as well as separately for each of the data sources. Normal prior distributions (N (0,1000)) were used for the regression parameters, and Cauchy (Cauchy (0, 25)) for the residual variance. Samples from the posterior distribution were obtained using the No-U-Turn Sampler with the Rstan package (Stan Development Team; https://mc-stan.org/). The underlying assumptions for normality of residuals and heteroskedasticity were assessed graphically. There were no violations of normality; assumptions of linearity were corrected with log transformation for dose concentration. Where appropriate, robust heteroskedastic consistent regression standard errors were used when there was evidence of non-equal variance. All analyses were undertaken in R Version 3.4.3 and SAS v9.4 (The SAS Institute; Carry, NC).

H scores (histo-scores; the outcome measures) for the three Groups at day 6 were compared using a linear mixed regression model which included a fixed effect for the treatment groups; the non-treated group was set as the reference. A random effect for each rat modelled the dependencies due to repeated measures. The subject number was used as the random effect allowing for a random intercept and dependent data. Robust standard errors were used to adjust for heteroscedasticity in the residuals.

### Digital Image Analysis

Whole slide digital images were acquired using an Aperio AT2 (Leica Biosystems, Wetzlar, Germany) at 20× magnification before analysis using the HALO v2.0 software (Indica laboratory, Corrales, New Mexico, USA). After selecting tissue areas, the percentage of total pixels with weak, moderate and strong staining intensity was measured using the ‘Area Quantification V1.0’ algorithm. Histology-score quantitation was calculated for each slide as H-score=(1×weak %)+(2×moderate %)+(3×strong %) providing a range from 0 to 300 (30). 4-6 sections per sample were scored at different levels through the tissue to obtain an average.

### Elisa Assay for Serum Heparan Sulphate

Serum samples, separated from whole blood were aliquoted and stored at −80° C. A commercial ELISA assay kit for heparan sulphate (CSB-E09585h) was then used according to the manufacturer's recommended protocol (Cusabio, Wuhan, China). Briefly, samples were first incubated with antibody for 30 min before addition of horseradish peroxidase conjugate solution for 30 min. Substrate was added for 20 min in the dark and the reaction stopped. All incubations were performed at 370 C. Absorbance was immediately measured at 450 nm on a Versa Max Microplate Reader (Molecular Devices, Sunnyvale, California, USA) and the results calculated in ng/ml according to the internal standard curve.

### Treatment

Castanospermine was administered by Alzet osmotic pumps (Alza Corporation, Palo Alto, United States) at a dose of 150 mg/kg per day.

### Results

The results show, surprisingly, that in both the allotransplant and the serum from allotransplanted rats, that Castanospermine effectively stops the breakdown of HSPG because the concentration of heparan sulphate (HS) in the Castanospermine-treated rats is not significantly different from the isograft control. Furthermore, in both these situations, the non-treated (rejecting) levels of HS are significantly higher than the isograft and significantly higher than the Castanospermine-treated rats. The isograft control is a control for ischaemia reperfusion injury (IRI). Hence, the data sets presented here exclude the effect of IRI and are limited to the effect of rejection with and without antirejection (Castanospermine) treatment.

Table 6 shows H-scores for three Groupwise comparisons. Three treatment Groups were studied at day 6 after transplantation. The results presented are the mean and 95% CI for each treatment Group; the means and 95% CI for the difference between them; p-value for the difference; and the Bayes Factors towards the alternative (BF₁₀) and towards the null (BF₀₁) hypotheses. H Score quantitation is calculated for each slide as H-score=(1×weak %)+(2×moderate %)+(3×strong %) giving a score ranging from 0 to 300. There were 4-6 slides per kidney sample from one rat scored (each taken at different levels through the tissue) to obtain an average for that rat. The Bayes Factor estimates how much evidence the data provides in support (or against) a null hypothesis.

Table 7 shows comparisons of serum heparan sulphate concentrations by treatment Group and by the study period, which is the combination of days 2, 4 and 6 after transplantation. The results presented are the ratios of the mean (estimate) and 95% CIs for each Group comparison for the combination of days 2, 4 and 6.

### DISCUSSION

The finding that Castanospermine effectively stops the breakdown of HSPG in the allotransplant and serum is unexpected and has major implications for treatment and prevention of allotransplant rejection. Any effect of ischaemia reperfusion injury is excluded by the use of the isograft control. The data means that Castanospermine, a heparanase inhibitor, has the effect of maintaining the integrity of the allotransplant despite it being subject to allotransplant rejection which destroys the transplant including its structure. Hence the use of heparanase inhibitors has application in induction, acute rejection and maintenance treatment in allotransplantation.

TABLE 6
H- scores for heparan sulphate in the renal allotransplant at
day 6 after transplantation using digital image pathology

| | | Estimate of | | | | | |
|---|---|---|---|---|---|---|---|
| Comparison | n | difference | LCL | UCL | P value | BF₁₀ | BFA₀₁ |
| Non-treated v | 75 | 31.70 | 15.43 | 47.96 | <0.001 | >100 | <0.01 |
| Isograft | | | | | | | |
| Non-treated v | 77 | 34.78 | 19.93 | 49.62 | <0.001 | >100 | <0.01 |
| Castanospermine | | | | | | | |
| Castanospermine v | 75 | −3.08 | −19.32 | 13.16 | 0.70 | <0.01 | >100 |
| Isograft | | | | | | | |

TABLE 7
Comparison of serum heparan sulphate levels for groups with
combined days 2, 4, 6 after renal allotransplantation

| | | Ratio of estimate | | | |
|---|---|---|---|---|---|
| Comparison | n | of difference | LCL | UCL | P value |
| Non-treated v | 74 | 1.81 | 1.46 | 2.23 | <0.001 |
| Isograft | | | | | |
| Non-treated v | 77 | 1.72 | 1.36 | 2.18 | <0.001 |
| Castanospermine | | | | | |
| Castanospermine v | 74 | 1.05 | 0.81 | 1.36 | 0.981 |
| Isograft | | | | | |

### EXAMPLES OF PREFERRED CLAIMS

A method of preventing or treating allotransplant rejection, said method comprising the step of administering to a subject in need thereof a heparanase inhibitor, wherein said heparanase inhibitor reduces the level of heparanase activity and thereby prevents or treats allotransplant rejection.

A method of maintaining allotransplant integrity, said method comprising the step of administering to a subject in need thereof a heparanase inhibitor, wherein said heparanase inhibitor reduces the level of heparanase activity and thereby maintains the integrity of the allotransplanted organ.

The method of [0169] or [0170], wherein said heparanase inhibitor reduces expression levels of heparanase in lymphocytes at the allotransplant site and wherein expression levels of heparanase in lymphocytes in the peripheral blood of the subject are not reduced.

The method according to any one of [0169] to [0171], wherein said allotransplant is a primarily vascularized organ transplant.

The method according to any one of [0169] to wherein [172] said allotransplant is a heart, kidney, lung, heart-lung, liver, pancreas, stomach or intestine transplant.

The method according to any one of [0169] to [0173], wherein said heparanase inhibitor is a benzoxazol-5-yl acetic acid derivative.

The method according to any one of [0169] to [0174], wherein said heparanase inhibitor is OGT 2115 having the formula,

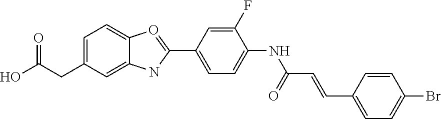

or a functional derivative or functional analog thereof.

The method according to any one of [0169] to [0173], wherein said heparanase inhibitor is Castanospermine.

The method according to any one of claims [0169] to [0176], wherein the heparanase inhibitor is administered in combination with one or more immunosuppressive drugs.

The method of [0177], wherein, the immunosuppressive drug is selected from the group consisting of methotrexate, mizoribine, cyclosporin, aerosolized cyclosporin, tacrolimus, mycophenolate mofetil, azathioprine, sirolimus and other mTOR inhibitors, deoxyspergualin, leflunomide, malononitriloamide analogs of leflunomide; anti-CTLA4 antibodies, anti-CTLA4 Ig fusions, anti-B lymphocyte stimulator antibodies, anti-CD80 antibodies, etanercept, infliximab, anti-T cell antibodies, anti-CD3 antibodies, OKT3, anti-CD4 antibodies, anti IL-2 receptor antibodies, prednisolone or its derivatives, anti-CD52 monoclonal antibodies; anti-CD20 monoclonal antibodies; belatacept; eculizumab; and intravenous immunoglobulin.

The method according to wherein the immunosuppressive drug is cyclosporin A or tacrolimus.

The method according to any of [0169] to [0176], wherein the heparanase inhibitor is administered in combination with one or more anti-inflammatory drugs.

The method of [0180], wherein the anti-inflammatory drug is selected from the group consisting of corticosteroids, clobetasol, halobetasol, hydrocortisone, triamcinolone, betamethasone, fluocinolone, fluocinonide, prednisone, prednisolone and methylprednisolone.

The method according to any one of [0169] to [0181], wherein said method prolongs the survival time of the allotransplant.

The method according to any one of [0169] to [0182], wherein said heparanase inhibitor is administered to said subject in a dose from 2.5 mg/kg to 10 mg/kg.

The method according to any one of [0169] to wherein said heparanase inhibitor is administered to said subject once daily.

The method according to any one of [0169] to [0184], wherein said heparanase inhibitor is administered to said subject once weekly.

The method of any one of [0170] to [0185], wherein transplantation integrity is maintained by substantially preventing heparan sulphate proteoglycan (HSPG) degradation by heparanase.

Use of a heparanase inhibitor in the preparation of a medicament for the prevention or treatment of allotransplant rejection wherein said heparanase inhibitor reduces the level of heparanase activity and thereby prevents or treats transplant rejection.

Use of a heparanase inhibitor in the preparation of a medicament for maintaining allotransplant integrity, wherein said heparanase inhibitor reduces the level of heparanase activity and thereby maintains transplantation integrity.

The use according to [187] or [188], wherein said heparanase inhibitor reduces expression levels of heparanase in lymphocytes at the allotransplant site and wherein expression levels of heparanase in lymphocytes in the peripheral blood are not reduced.

The use according to any one of [187] to [189] wherein said allotransplant is a primarily vascularized organ transplant.

The use according to any one of claims [187] to [190] wherein said allotransplant is a heart, kidney, lung, heart-lung, liver, pancreas, stomach or intestine transplant.

The use according to any one of claims [187] to [191], wherein said heparanase inhibitor is a benzoxazol-5-yl acetic acid derivative.

The use according to any one of claims [187] to [192], wherein said heparanase inhibitor is OGT 2115 having the formula,

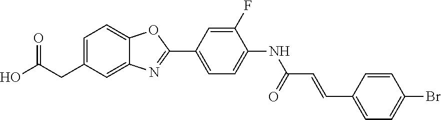 [SMILES: O=C(O)Cc1ccc2oc(-c3ccc(NC(=O)/C=C/c4ccc(Br)cc4)c(F)c3)nc2c1]

or a functional derivative or functional analog thereof.

The use according to any one of [187] to [191] wherein said heparanase inhibitor is Castanospermine.

The use according to any one of [187] to [194], wherein the heparanase inhibitor is formulated for administration in combination with one or more immunosuppressive drugs.

The use according to [195], wherein, the immunosuppressive drug is selected from the group consisting of methotrexate, mizoribine, cyclosporin, aerosolized cyclosporin, tacrolimus, mycophenolate mofetil, azathioprine, sirolimus and other mTOR inhibitors, deoxyspergualin, leflunomide, malononitriloamide analogs of leflunomide; anti-CTLA4 antibodies, anti-CTLA4 Ig fusions, anti-B lymphocyte stimulator antibodies, anti-CD80 antibodies, etanercept, infliximab, anti-T cell antibodies, anti-CD3 antibodies, OKT3, anti-CD4 antibodies, anti il-2 receptor antibodies, prednisolone or its derivatives, anti-CD52 monoclonal antibodies; anti-CD20 monoclonal antibodies; belatacept; eculizumab; and intravenous immunoglobulin.

The use according to [196] wherein the immunosuppressive drug is cyclosporin A or tacrolimus.

The use according to any one of claims [187] to [194], wherein the heparanase inhibitor is formulated for administration in combination with one or more anti-inflammatory drugs.

The use according to any one of [198], wherein the anti-inflammatory drug is selected from the group consisting of corticosteroids, clobetasol, halobetasol, hydrocortisone, triamcinolone, betamethasone, fluocinolone, fluocinonide, prednisone, prednisolone and methylprednisolone.

The use according to any one of claims [187] to [199], wherein said use prolongs the survival time of a transplant.

The use of any one of claims [188] to [200], wherein transplantation integrity is maintained by substantially preventing heparan sulphate proteoglycan (HSPG) degradation by heparanase.

### REFERENCES

1. WO 2018/107201 (the Australian National University) May 2018 pages 125, 127, 144.
2. Masola V, Bellin G, Vischini G et al. Inhibition of heparanase protects against chronic kidney dysfunction following ischaemia reperfusion injury. Oncotarget 2018; 9(90): 36185-201.
3. Noda K et al. Heparanase inhibition preserves the endothelial glycocalyx in lung grafts and improves lung preservation and transplant outcomes. Sci Rep. 2021 Jun. 10; 11(1): 12265. doi: 10.1038/s41598-021-91777-0.
4. Song W Y et al. Inhibition of heparanase protects against pancreatic beta cell death in streptozotocin-induced diabetic mice via reducing intra-islet inflammatory cell infiltration. Br J Pharmacol. 2020 October; 177(19): 4433-4447. doi: 10.1111/bph. 15183. Epub 2020 Aug. 19. PMID: 32608014; PMCID: PMC7484558
5. WO 2011/109877 A (The Australian National University 15 Sep. 2011
6. WO 2008/046162 (The Australian National University 24 Apr. 2008)
7. Coombe and Gandhi Heparanase: A Challenging Cancer Drug Target. Front. Oncol. 28 Nov. 2019.
8. Lider O et al. Suppression of experimental autoimmune diseases and prolongation of allograft survival by treatment of animals with low doses of heparins. J Clin Invest. 1989 March; 83(3): 752-6. doi: 10.1172/JCI113953.
9. Cohen A|CA 1318591 1 June 199310. Cahalon, L, Lider, O, Schor, H et al. Heparin disaccharides inhibit tumor necrosis factor-α production by macrophages and arrest immune inflammation in rodents. Int. Immunol. 9:1517, 1997.
10. Ono K, Lindsey E S. Improved technique of heart transplantation in rats. J Thorac Cardiovasc Surg 1969; 57:225-229 [PMID: 4884735]
11. Lee S. An improved technique of renal transplantation in the rat. Surgery. 1967; 61(5): 771-773.

1. A method of preventing or treating allotransplant rejection,
said method comprising the step of administering to a subject in need thereof a heparanase inhibitor, wherein said heparanase inhibitor reduces expression levels of heparanase in lymphocytes at the allotransplant site and wherein expression levels of heparinase in lymphocytes in the peripheral blood of the subject are not reduced, thereby preventing or treating allotransplant rejection, and wherein:
said allotransplant is a primarily vascularized organ transplant;
said heparanase inhibitor excludes castanospermine, and
the primarily vascularized organ transplant excludes skin.

2. A method of maintaining allotransplant integrity, said method comprising the step of administering to a subject in need thereof a heparanase inhibitor, wherein said heparanase inhibitor reduces expression levels of heparanase in lymphocytes at the allotransplant site and wherein expression levels of in lymphocytes in the peripheral blood of the subject are not reduced, thereby maintaining transplantation integrity, and wherein:
said allotransplant is a primarily vascularized organ transplant
said inhibitor excludes castanospermine; and
the primarily vascularized organ transplant excludes skin.

3. (canceled)

4. (canceled)

5. The method according to claim 1 wherein said allotransplant is a heart, kidney, lung, liver, pancreas, stomach or intestine transplant.

6. (canceled)

7. The method according to claim 1, wherein said heparanase inhibitor is OGT 2115 having the formula, 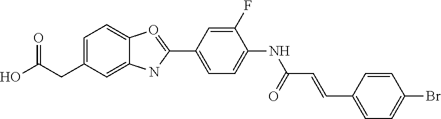
or a functional derivative or functional analog thereof.

8. (canceled)

9. The method according to claim 1, wherein the heparanase inhibitor is administered in combination with one or more immunosuppressive drugs or one or more anti-inflammatory drugs.

10. The method according to claim 9, wherein, the immunosuppressive drug is selected from the group consisting of methotrexate, mizoribine, cyclosporin, aerosolized cyclosporin, tacrolimus, mycophenolate mofetil, azathioprine, sirolimus and other mTOR inhibitors, deoxyspergualin, leflunomide, malononitriloamide analogs of leflunomide; anti-CTLA4 antibodies, anti-CTLA4 Ig fusions, anti-B lymphocyte stimulator antibodies, anti-CD80 antibodies, etanercept, infliximab, anti-T cell antibodies, anti-CD3 antibodies, OKT3, anti-CD4 antibodies, anti IL-2 receptor antibodies, prednisolone or its derivatives, anti-CD52 monoclonal antibodies; anti-CD20 monoclonal antibodies; belatacept; eculizumab; and intravenous immunoglobulin.

11. The method according to claim 9 wherein the immunosuppressive drug is cyclosporin A or tacrolimus.

12. The method according to claim 9, wherein the heparanase inhibitor is administered together or sequentially with the one or more immunosuppressive drugs or one or more anti-inflammatory drugs.

13. The method of claim 12, wherein the anti-inflammatory drug is selected from the group consisting of corticosteroids, clobetasol, halobetasol, hydrocortisone, triamcinolone, betamethasone, fluocinolone, fluocinonide, prednisone, prednisolone and methylprednisolone.

14. The method according to claim 1, wherein said heparanase inhibitor is administered to said subject in a dose from 2.5 mg/kg to 50 mg/kg.

15. (canceled)

16. The method according to claim 1, wherein said heparanase inhibitor is administered to said subject once daily or once weekly.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The method according to claim 2 wherein said allotransplant is a heart, kidney, lung, liver, pancreas, stomach or intestine transplant.

35. The method according to claim 2, wherein said heparanase inhibitor is OGT 2115 having the formula, 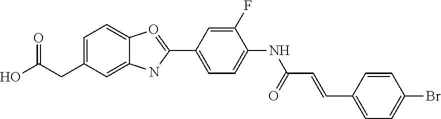
or a functional derivative or functional analog thereof.

36. The method according to claim 2, wherein the heparanase inhibitor is administered in combination with one or more immunosuppressive drugs or one or more anti-inflammatory drugs.

37. The method according to claim 9, wherein, the immunosuppressive drug is selected from the group consisting of methotrexate, mizoribine, cyclosporin, aerosolized cyclosporin, tacrolimus, mycophenolate mofetil, azathioprine, sirolimus and other mTOR inhibitors, deoxyspergualin, leflunomide, malononitriloamide analogs of leflunomide; anti-CTLA4 antibodies, anti-CTLA4 Ig fusions, anti-B lymphocyte stimulator antibodies, anti-CD80 antibodies, etanercept, infliximab, anti-T cell antibodies, anti-CD3 antibodies, OKT3, anti-CD4 antibodies, anti IL-2 receptor antibodies, prednisolone or its derivatives, anti-CD52 monoclonal antibodies; anti-CD20 monoclonal antibodies; belatacept; eculizumab; and intravenous immunoglobulin.

38. The method according to claim 37 wherein the immunosuppressive drug is cyclosporin A or tacrolimus.

39. The method according to claim 37, wherein the heparanase inhibitor and one or more anti-inflammatory drugs or one or more immunosuppressive drugs are administered together or sequentially.

40. The method of claim 39 wherein the anti-inflammatory drug is selected from the group consisting of corticosteroids, clobetasol, halobetasol, hydrocortisone, triamcinolone, betamethasone, fluocinolone, fluocinonide, prednisone, prednisolone and methylprednisolone.

41. The method according to claim 2, wherein said heparanase inhibitor is administered to said subject in a dose from 2.5 mg/kg to 50 mg/kg.

42. The method according to claim 2, wherein said heparanase inhibitor is administered to said subject once daily or once weekly.
